SEQUENCE LISTING (17 sequences)

SEQ ID NO: 1 - protein, 28 aa, Homo Sapiens
GSSFLSPEHQRVQQRKESKKPPAKLQPR

SEQ ID NO: 2 - protein, 27 aa, Homo Sapiens
GSSFLSPEHQRVQQRKESKKPPAKLQP

SEQ ID NO: 3 - protein, 23 aa, Homo Sapiens
FNAPFDVGIKLSGVQYQQHSQAL

SEQ ID NO: 4 - protein, 58 aa, Homo Sapiens
VSQRTDGESRAHLGALLARYIQQARKAPSGRMSIVKNLQNLDPSHRISDRDYMGWMDF

SEQ ID NO: 5 - protein, 30 aa, Homo Sapiens
HAEGTFTSDVSSYLEGQAAKEFIAWLVKGR

SEQ ID NO: 6 - protein, 36 aa, Homo Sapiens
YPSKPDNPGEDAPAEDMARYYSALRHYINLITRQRY

SEQ ID NO: 7 - protein, 13 aa, Homo Sapiens
  Acetylation (1)..(1)
SYSMEHFRWGKPV

SEQ ID NO: 8 - DNA, 552 bp, Homo Sapiens
acctccgccaggaactgcaggcccacctgtctgcaacccagctgaggccatgccctccccagggaccgtctgcagcctcctgctcctcggcatgctctggctggacttggccatggcaggctccagcttcctgagccctgaacaccagagagtccagcagagaaaggagtcgaagaagccaccagccaagctgcagccccgagctctagcaggctggctccgcccggaagatggaggtcaagcagaaggggcagaggatgaactggaagtccggttcaacgccccctttgatgttggaatcaagctgtcaggggttcagtaccagcagcacagccaggccctggggaagtttcttcaggacatcctctgggaagaggccaaagaggccccagccgacaagtgatcgcccacaagccttactcacctctctctaagtttagaagcgctcatctggcttttcgcttgcttctgcagcaactcccacgactgttgtacaagctcaggaggcgaataaatgttcaaactgtaaaaaaaaaaaaaaaaaaaaaaaaa

SEQ ID NO: 9 - DNA, 851 bp, Homo Sapiens
cacttcaaccggttgtcgccccagtggccgccctctgagcacgtgttactgccagtctgcgtcagcgttgggtaaatacatgactggccgacgccgccgggcggggctatttaagagacagccgcccgctggtcctccctgaacttggctcagctgccgggctgctccggttggaaacgccaagccagctgcgtcctaatccaaaagccatgaacagcggcgtgtgcctgtgcgtgctgatggcggtactggcggctggcgccctgacgcagccggtgcctcccgcagatcccgcgggctccgggctgcagcgggcagaggaggcgccccgtaggcagctgagggtatcgcagagaacggatggcgagtcccgagcgcacctgggcgccctgctggcaagatacatccagcaggcccggaaagctccttctggacgaatgtccatcgttaagaacctgcagaacctggaccccagccacaggataagtgaccgggactacatgggctggatggattttggccgtcgcagtgccgaggagtatgagtacccctcctagaggacccagccgccatcagcccaacgggaagcaacctcccaacccagaggaggcagaataagaaaacaatcacactcataactcattgtctgtggagtttgacattgtatgtatctatttattaagttctcaatgtgaaaaatgtgtctgtaagattgtccagtgcaaccacacacctcaccagaattgtgcaaatggaagacaaaatgttttcttcatctgtgactcctggtctgaaaatgttgttatgctattaaagtgatttcattctgaaaaaaaaaaaaaaaaaaaaaa

SEQ ID NO: 10 - DNA, 1128 bp, Homo Sapiens
acagagcttaggacacagagcacatcaaaagttcccaaagagggcttgctctctcttcacctgctctgttctacagcacactaccagaagacagcagaaatgaaaagcatttactttgtggctggattatttgtaatgctggtacaaggcagctggcaacgttcccttcaagacacagaggagaaatccagatcattctcagcttcccaggcagacccactcagtgatcctgatcagatgaacgaggacaagcgccattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagacatgctgatggttctttctctgatgagatgaacaccattcttgataatcttgccgccagggactttataaactggttgattcagaccaaaatcactgacaggaaataactatatcactattcaagatcatcttcacaacatcacctgctagccacgtgggatgtttgaaatgttaagtcctgtaaatttaagaggtgtattctgaggccacattgctttgcatgccaataaataaattttcttttagtgttgtgtagccaaaaattacaaatggaataaagttttatcaaaatattgctaaaatatcagctttaaaatatgaaagtgctagattctgttattttcttcttattttggatgaagtaccccaacctgtttacatttagcgataaaattatttttctatgatataatttgtaaatgtaaattattccgatctgacatatctgcattataataataggagaatagaagaactggtagccacagtggtgaaattggaaagagaactttcttcctgaaacctttgtcttaaaaatactcagctttcaatgtatcaaagatacaattaaataaaattttcaagcttcttta

SEQ ID NO: 11 - DNA, 576 bp, Homo Sapiens
gcaccccatccgctggctctcacccctcggagacgctcgcccgacagcatagtacttgccgcccagccacgcccgcgcgccagccaccatgctaggtaacaagcgactggggctgtccggactgaccctcgccctgtccctgctcgtgtgcctgggtgcgctggccgaggcgtacccctccaagccggacaacccgggcgaggacgcaccagcggaggacatggccagatactactcggcgctgcgacactacatcaacctcatcaccaggcagagatatggaaaacgatccagcccagagacactgatttcagacctcttgatgagagaaagcacagaaaatgttcccagaactcggcttgaagaccctgcaatgtggtgatgggaaatgagacttgctctctggccttttcctattttcagcccatatttcatcgtgtaaaacgagaatccacccatcctaccaatgcatgcagccactgtgctgaattctgcaatgttttcctttgtcatcattgtatatatgtgtgtttaaataaagtatcatgcattcaaaagtgaaaaaaaaaaaaaaaa

SEQ ID NO: 12 - DNA, 1245 bp, Homo Sapiens
ccttcccctggcccggggagctgctccttgtgctgccgggaaggtcaaagtcccgcgcccaccaggagagctcggcaagtatataaggacagaggagcgcgggaccaagcggcggcgaaggaggggaagaagagccgcgaccgagagaggccgccgagcgtccccgccctcagagagcagcctcccgagacagagcctcagcctgcctggaagatgccgagatcgtgctgcagccgctcgggggccctgttgctggccttgctgcttcaggcctccatggaagtgcgtggctggtgcctggagagcagccagtgtcaggacctcaccacggaaagcaacctgctggagtgcatccgggcctgcaagcccgacctctcggccgagactcccatgttcccgggaaatggcgacgagcagcctctgaccgagaacccccggaagtacgtcatgggccacttccgctgggaccgattcggccgccgcaacagcagcagcagcggcagcagcggcgcagggcagaagcgcgaggacgtctcagcgggcgaagactgcggcccgctgcctgagggcggccccgagccccgcagcgatggtgccaagccgggcccgcgcgagggcaagcgctcctactccatggagcacttccgctggggcaagccggtgggcaagaagcggcgcccagtgaaggtgtaccctaacggcgccgaggacgagtcggccgaggccttccccctggagttcaagagggagctgactggccagcgactccgggagggagatggccccgacggccctgccgatgacggcgcaggggcccaggccgacctggagcacagcctgctggtggcggccgagaagaaggacgagggcccctacaggatggagcacttccgctggggcagcccgcccaaggacaagcgctacggcggtttcatgacctccgagaagagccagacgcccctggtgacgctgttcaaaaacgccatcatcaagaacgcctacaagaagggcgagtgagggcacagcggggccccagggctaccctcccccaggaggtcgaccccaaagccccttgctctcccctgccctgctgccgcctcccagcctggggggtcgtggcagataatcagcctcttaaagctgcctgtagttaggaaataaaacctttcaaatttcacatccacctctgactttgaatgtaaactgtgtgaataaagtaaaaatacgtagccgtcaaataacagc

SEQ ID NO: 13 - protein, 117 aa, Homo Sapiens
MPSPGTVCSLLLLGMLWLDLAMAGSSFLSPEHQRVQQRKESKKPPAKLQPRALAGWLRPEDGGQAEGAEDELEVRFNAPFDVGIKLSGVQYQQHSQALGKFLQDILWEEAKEAPADK

SEQ ID NO: 14 - protein, 115 aa, Homo Sapiens
MNSGVCLCVLMAVLAAGALTQPVPPADPAGSGLQRAEEAPRRQLRVSQRTDGESRAHLGALLARYIQQARKAPSGRMSIVKNLQNLDPSHRISDRDYMGWMDFGRRSAEEYEYPS

SEQ ID NO: 15 - protein, 180 aa, Homo Sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 16 - protein, 97 aa, Homo Sapiens
MLGNKRLGLSGLTLALSLLVCLGALAEAYPSKPDNPGEDAPAEDMARYYSALRHYINLITRQRYGKRSSPETLISDLLMRESTENVPRTRLEDPAMW

SEQ ID NO: 17 - protein, 267 aa, Homo Sapiens
MPRSCCSRSGALLLALLLQASMEVRGWCLESSQCQDLTTESNLLECIRACKPDLSAETPMFPGNGDEQPLTENPRKYVMGHFRWDRFGRRNSSSSGSSGAGQKREDVSAGEDCGPLPEGGPEPRSDGAKPGPREGKRSYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFMTSEKSQTPLVTLFKNAIIKNAYKKGE

(19) United States
(12) Patent Application Publication
(10) Pub. No.: US 2010/0210541 A1
(43) Pub. Date: Aug. 19, 2010

(54) Biomarkers for Appetite Regulation

(76) Inventor: Andreas Pfuetzner, Mainz (DE)

Correspondence Address: MORGAN, LEWIS & BOCKIUS, LLP (SF), ONE MARKET SPEAR STREET TOWER, SAN FRANCISCO, CA, 94105, US

(21) Appl. No.: 12/684,058

(22) Filed: Jan. 7, 2010

Related U.S. Application Data
(60) Provisional application No. 61/143,059, filed on Jan. 7, 2009.

(51) Int. Cl. A61K 38/16; A61P 3/04; A61P 3/00; C40B 40/10; G01N 33/68; C40B 30/04

(52) U.S. Cl. 514/12; 506/18; 436/86; 506/9

(57) ABSTRACT

The invention provides compositions and methods for characterizing appetite regulation in a subject. In one embodiment, the composition comprises a solid support comprising probes for measuring a biomarker panel comprising, for example, total ghrelin, obestatin, cholecystokinin, GLP-1(6-37)-NH₂, NPY and α-MSH. The simultaneous use of multiple biomarkers with independent classification power will increase the performance of the biomarker panel in characterizing appetite regulation. The invention also provides methods of treating a subject (e.g. one experiencing obesity) and determining the efficacy of a therapy through assaying the various biomarkers of a biomarker panel disclosed herein.

### CROSS-REFERENCE TO RELATED APPLICATIONS

This application claims under 35 USC 119(e) the benefit of U.S. Application 61/143,059, filed Jan. 7, 2009, which is incorporated by reference its entirety.

### REFERENCE TO SEQUENCE LISTING SUBMITTED ELECTRONICALLY

The sequence listing contained in the file named “12684058seqlist.txt”, created on Apr. 12, 2010 and having a size of 16 kilobytes, has been submitted electronically herewith via EFS-Web, and the contents of the txt file are hereby incorporated by reference in their entirety.

### TECHNICAL FIELD

The invention provides compositions and methods for characterizing appetite regulation in a subject. The invention also provides compositions and methods for treating a subject and determining the efficacy of a therapy according to levels of biomarkers associated with appetite regulation.

### BACKGROUND

Current methods of assessing the effectiveness of appetite regulation rely on measurements of body weight. Simple assessment of body weight, however, does not elucidate the complex mechanisms of satiety regulation that is required to understand drug mechanisms.

There thus remains a need for a means of determining a subject's appetite regulation. Such means is provided by the present invention.

### SUMMARY OF INVENTION

In one aspect, the invention provides a kit comprising a solid support comprising: (a) a capture binding ligand selective for total ghrelin, (b) a capture binding ligand selective for obestatin, (c) a capture binding ligand selective for cholecystokinin, (d) a capture binding ligand selective for GLP-1(6-37)-NH₂, (e) a capture binding ligand selective for NPY and (f) a capture binding ligand selective for α-MSH.

In one embodiment, one of the capture binding ligands comprises an antibody.

In one embodiment, the kit further comprises: (a) a soluble binding ligand selective for total ghrelin, (b) a soluble binding ligand selective for obestatin, (c) a soluble binding ligand selective for cholecystokinin, (d) a soluble binding ligand selective for GLP-1(6-37)-NH₂, (e) a soluble binding ligand selective for NPY and (f) a soluble binding ligand selective for α-MSH, wherein each of the soluble capture ligands comprises a detectable label.

In one embodiment, a detectable label is a fluorophore.

In one embodiment, a detectable label comprises biotin.

In one embodiment, the kit further comprises a horseradish peroxidase conjugate.

In one embodiment, the kit further comprises a precipitating agent.

In one aspect, the invention provides a method of assaying a sample comprising (a) taking a measurement of the concentrations of total ghrelin, obestatin, cholecystokinin, GLP-1(6-37)-NH₂, NPY and α-MSH, thereby assaying the sample.

In one embodiment, the sample is derived from a subject.

In one aspect, the invention provides a method of treating disease in a subject comprising: (a) measuring the concentrations of total ghrelin, obestatin, cholecystokinin, GLP-1(6-37)-NH₂, NPY and α-MSH in a first sample from the subject; and (b) effecting a first therapy on the subject, wherein one, a combination or all of the concentrations of total ghrelin, obestatin, cholecystokinin, GLP-1(6-37)-NH₂, NPY and α-MSH in a second sample from the subject are changed with respect to the first sample.

In one embodiment, one, a combination or all of the changes selected from (a) a decrease in total ghrelin concentration, (b) an increase in obestatin concentration, (c) an increase in cholecystokinin concentration, (d) an increase in GLP-1(6-37)-NH₂ concentration, (e) a decrease in NPY concentration and (f) an increase in α-MSH concentration occur(s) between the first sample and the second sample from the subject after the first therapy.

In one embodiment, one, a combination or all of the changes selected from (a) a decrease in total ghrelin concentration to below about 5 μg/L, (b) an increase in obestatin concentration to above about 40 ng/L, (c) an increase in cholecystokinin concentration to above about 1 μg/L, (d) an increase in GLP-1(6-37)-NH₂ concentration to above about 20 pg/mL, (e) a decrease in NPY concentration to below about 10 pmol/L and (f) an increase in α-MSH concentration to above about 20 ng/L occur(s) between the first sample and the second sample from the subject after the first therapy.

In one embodiment, in the second sample, total ghrelin concentration is below about 5 μg/L, obestatin concentration is above about 40 ng/L, cholecystokinin concentration is above about 1 μg/L, GLP-1(6-37)-NH₂ concentration is above about 20 pg/mL, NPY concentration is below about 10 pmol/L and α-MSH concentration is above about 20 ng/L.

In one embodiment, effecting the first therapy comprises administering a first disease-modulating drug to the subject, optionally wherein the drug is a GLP-1 analog.

In one embodiment, effecting the first therapy comprises causing the subject to follow a dietary regimen having a high fiber and low carbohydrate content.

In one aspect, the invention provides a method of assessing the efficacy of a first therapy on a subject comprising: (a) taking a first measurement of the concentrations of total ghrelin, obestatin, cholecystokinin, GLP-1(6-37)-NH₂, NPY and α-MSH in a first sample from the subject; (b) effecting the first therapy on the subject; (c) taking a second measurement of the concentrations of total ghrelin, obestatin, cholecystokinin, GLP-1(6-37)-NH₂, NPY and α-MSH in a second sample from the subject; and (d) making a comparison between the first and second measurements.

In one embodiment, the method further comprises (e) effecting a second therapy on the subject based on the comparison.

In one embodiment, effecting the first therapy comprises administering a first disease-modulating drug to the subject according to a first dosage regimen.

In one embodiment, effecting the second therapy comprises making a decision regarding the continued administration of the first disease-modulating drug.

In one embodiment, effecting the second therapy comprises administering a second disease-modulating drug to the subject.

In one embodiment, effecting the second therapy comprises administering a statin to the subject.

In one embodiment, effecting the second therapy comprises discontinuing the administration of the first disease-modulating drug.

In one embodiment, effecting the second therapy comprises repeating or maintaining the administration of the first disease-modulating drug.

In one embodiment, effecting the second therapy comprises administering the first disease-modulating drug according to an adjusted dosage regimen compared to the first dosage regimen.

In one embodiment, the adjusted dosage regimen depends on the degree of change in the concentration(s) of one, a combination or all of total ghrelin, obestatin, cholecystokinin, GLP-1(6-37)-NH₂, NPY and α-MSH between the first and second measurement.

In one embodiment, if one, a combination or all of the changes selected from (a) a decrease in total ghrelin concentration, (b) an increase in obestatin concentration, (c) an increase in cholecystokinin concentration, (d) an increase in GLP-1(6-37)-NH₂ concentration, (e) a decrease in NPY concentration and (f) an increase in α-MSH concentration occur(s) between the first and second measurements, then effecting the second therapy comprises repeating or maintaining the administration of the first disease-modulating drug.

In one embodiment, if one, a combination or all of the changes selected from (a) a decrease in total ghrelin concentration to below about 5 μg/L, (b) an increase in obestatin concentration to above about 40 ng/L, (c) an increase in cholecystokinin concentration to above about 1 μg/L, (d) an increase in GLP-1(6-37)-NH₂ concentration to above about 20 pg/mL, (e) a decrease in NPY concentration to below about 10 pmol/L and (f) an increase in α-MSH concentration to above about 20 ng/L occur(s) between the first and second measurements, then effecting the second therapy comprises repeating or maintaining the administration of the first disease-modulating drug.

In one embodiment, if one, a combination or all of the changes selected from (a) a decrease in total ghrelin concentration, (b) an increase in obestatin concentration, (c) an increase in cholecystokinin concentration, (d) an increase in GLP-1(6-37)-NH₂ concentration, (e) a decrease in NPY concentration and (f) an increase in α-MSH concentration do(es) not occur between the first and second measurements, then effecting the second therapy comprises discontinuing the administration of the first disease-modulating drug.

In one embodiment, if one, a combination or all of the changes selected from (a) a decrease in total ghrelin concentration to below about 5 μg/L, (b) an increase in obestatin concentration to above about 40 ng/L, (c) an increase in cholecystokinin concentration to above about 1 μg/L, (d) an increase in GLP-1(6-37)-NH₂ concentration to above about 20 pg/mL, (e) a decrease in NPY concentration to below about 10 pmol/L and (f) an increase in α-MSH concentration to above about 20 ng/L do(es) not occur between the first and second measurements, then effecting the second therapy comprises discontinuing the administration of the first disease-modulating drug.

In one embodiment, the first disease-modulating drug is a GLP-1 analog.

In one embodiment, effecting the first therapy comprises causing the subject to follow a dietary regimen having a high fiber and low carbohydrate content.

In one embodiment, one, a combination or all of the changes selected from (a) a decrease in total ghrelin concentration, (b) an increase in obestatin concentration, (c) an increase in cholecystokinin concentration, (d) an increase in GLP-1(6-37)-NH₂ concentration, (e) a decrease in NPY concentration and (f) an increase in α-MSH concentration occur(s) between the first and second measurements.

In one embodiment, one, a combination or all of the changes selected from (a) a decrease in total ghrelin concentration to below about 5 μg/L, (b) an increase in obestatin concentration to above about 40 ng/L, (c) an increase in cholecystokinin concentration to above about 1 μg/L, (d) an increase in GLP-1(6-37)-NH₂ concentration to above about 20 pg/mL, (e) a decrease in NPY concentration to below about 10 pmol/L and (f) an increase in α-MSH concentration to above about 20 ng/L occur(s) between the first and second measurements.

In one embodiment, one, a combination or all of the changes selected from (a) a decrease in total ghrelin concentration, (b) an increase in obestatin concentration, (c) an increase in cholecystokinin concentration, (d) an increase in GLP-1(6-37)-NH₂ concentration, (e) a decrease in NPY concentration and (f) an increase in α-MSH concentration do(es) not occur between the first and second measurements.

In one embodiment, one, a combination or all of the changes selected from (a) a decrease in total ghrelin concentration to below about 5 μg/L, (b) an increase in obestatin concentration to above about 40 ng/L, (c) an increase in cholecystokinin concentration to above about 1 μg/L, (d) an increase in GLP-1(6-37)-NH₂ concentration to above about 20 pg/mL, (e) a decrease in NPY concentration to below about 10 pmol/L and (f) an increase in α-MSH concentration to above about 20 ng/L do(es) not occur between the first and second measurements.

In one embodiment, the method further comprises effecting a second therapy comprising administering a disease modulating drug to the subject, optionally wherein the drug is a GLP-1 analog.

In one embodiment, the subject is experiencing obesity.

In one embodiment, a sample comprises plasma or serum.

In one embodiment, a sample is contacted with the solid support of a kit of the invention.

In one aspect, the invention provides a method of acquiring data relating to a sample comprising (a) taking a measurement of the concentrations of total ghrelin, obestatin, cholecystokinin, GLP-1(6-37)-NH₂, NPY and α-MSH in the sample, thereby acquiring data relating to the sample.

In one embodiment, the sample is derived from a subject, optionally wherein the subject is experiencing obesity.

In one embodiment, the sample comprises plasma or serum.

In one embodiment, the sample is contacted with the solid support of a kit of the invention.

In one aspect, the invention provides use of a kit of the invention to determine a second therapy for a subject that has undergone a first therapy, wherein the subject is experiencing obesity.

In one aspect, the invention provides use of the kit of the invention to determine whether a subject belongs to a population that would benefit from a second therapy, wherein the subject has undergone a first therapy.

In one embodiment, the use comprises (a) contacting a first sample from the subject with the solid support of the kit; (b) taking a first measurement of the concentrations of total ghrelin, obestatin, cholecystokinin, GLP-1(6-37)-NH₂, NPY and α-MSH in the first sample; (c) effecting a first therapy on the subject; (d) contacting a second sample from the subject with the solid support of the kit; (e) taking a second measurement of the concentrations of total ghrelin, obestatin, cholecystokinin, GLP-1(6-37)-NH₂, NPY and α-MSH in the second sample; and (f) making a comparison of the first and second measurements.

In one embodiment, the first therapy comprises administering a first disease-modulating drug to the subject according to a first dosage regimen.

In one embodiment, the second therapy comprises administering a second disease-modulating drug to the subject.

In one embodiment, the second therapy comprises administering a statin to the subject.

In one embodiment, the second therapy comprises discontinuing the administration of the first disease-modulating drug.

In one embodiment, the second therapy comprises repeating or maintaining the administration of the first disease-modulating drug.

In one embodiment, the second therapy comprises administering the first disease-modulating drug according to an adjusted dosage regimen compared to the first dosage regimen.

In one embodiment, the adjusted dosage regimen depends on the degree of change in the concentration(s) of one, a combination or all of total ghrelin, obestatin, cholecystokinin, GLP-1(6-37)-NH₂, NPY and α-MSH between the first and second measurement.

In one embodiment, if one, a combination or all of the changes selected from (a) a decrease in total ghrelin concentration, (b) an increase in obestatin concentration, (c) an increase in cholecystokinin concentration, (d) an increase in GLP-1(6-37)-NH₂ concentration, (e) a decrease in NPY concentration and (f) an increase in α-MSH concentration occur(s) between the first and second measurements, then the second therapy comprises repeating or maintaining the administration of the first disease-modulating drug.

In one embodiment, if one, a combination or all of the changes selected from (a) a decrease in total ghrelin concentration to below about 5 μg/L, (b) an increase in obestatin concentration to above about 40 ng/L, (c) an increase in cholecystokinin concentration to above about 1 μg/L, (d) an increase in GLP-1(6-37)-NH₂ concentration to above about 20 pg/mL, (e) a decrease in NPY concentration to below about 10 pmol/L and (f) an increase in α-MSH concentration to above about 20 ng/L occur(s) between the first and second measurements, then the second therapy comprises repeating or maintaining the administration of the first disease-modulating drug.

In one embodiment, if one, a combination or all of the changes selected from (a) a decrease in total ghrelin concentration, (b) an increase in obestatin concentration, (c) an increase in cholecystokinin concentration, (d) an increase in GLP-1(6-37)-NH₂ concentration, (e) a decrease in NPY concentration and (f) an increase in α-MSH concentration occur(s) between the first and second measurements do(es) not occur between the first and second measurements, then the second therapy comprises discontinuing the administration of the first disease-modulating drug.

In one embodiment, if one, a combination or all of the changes selected from (a) a decrease in total ghrelin concentration to below about 5 μg/L, (b) an increase in obestatin concentration to above about 40 ng/L, (c) an increase in cholecystokinin concentration to above about 1 μg/L, (d) an increase in GLP-1(6-37)-NH₂ concentration to above about 20 pg/mL, (e) a decrease in NPY concentration to below about 10 pmol/L and (f) an increase in α-MSH concentration to above about 20 ng/L do(es) not occur between the first and second measurements, then the second therapy comprises discontinuing the administration of the first disease-modulating drug.

In one embodiment, the first disease-modulating drug is a GLP-1 analog.

In one embodiment, the first therapy comprises causing the subject to follow a dietary regimen having a high fiber and low carbohydrate content.

In one embodiment, one, a combination or all of the changes selected from (a) a decrease in total ghrelin concentration, (b) an increase in obestatin concentration, (c) an increase in cholecystokinin concentration, (d) an increase in GLP-1(6-37)-NH₂ concentration, (e) a decrease in NPY concentration and (f) an increase in α-MSH concentration occur(s) between the first and second measurements.

In one embodiment, one, a combination or all of the changes selected from (a) a decrease in total ghrelin concentration to below about 5 μg/L, (b) an increase in obestatin concentration to above about 40 ng/L, (c) an increase in cholecystokinin concentration to above about 1 μg/L, (d) an increase in GLP-1(6-37)-NH₂ concentration to above about 20 pg/mL, (e) a decrease in NPY concentration to below about 10 pmol/L and (f) an increase in α-MSH concentration to above about 20 ng/L occur(s) between the first and second measurements.

In one embodiment, one, a combination or all of the changes selected from (a) a decrease in total ghrelin concentration, (b) an increase in obestatin concentration, (c) an increase in cholecystokinin concentration, (d) an increase in GLP-1(6-37)-NH₂ concentration, (e) a decrease in NPY concentration and (f) an increase in α-MSH concentration do(es) not occur between the first and second measurements.

In one embodiment, one, a combination or all of the changes selected from (a) a decrease in total ghrelin concentration to below about 5 μg/L, (b) an increase in obestatin concentration to above about 40 ng/L, (c) an increase in cholecystokinin concentration to above about 1 μg/L, (d) an increase in GLP-1(6-37)-NH₂ concentration to above about 20 pg/mL, (e) a decrease in NPY concentration to below about 10 pmol/L and (f) an increase in α-MSH concentration to above about 20 ng/L do(es) not occur between the first and second measurements.

In one embodiment, the second therapy comprises administering a disease modulating drug to the subject, optionally wherein the drug is a GLP-1 analog.

In one embodiment, the subject is experiencing obesity.

In one embodiment, a sample comprises plasma or serum.

In one embodiment, a given biomarker panel can be replaced with any other panel disclosed herein.

### BRIEF DESCRIPTION OF DRAWINGS

FIG. 1 shows examples of two different assay configurations.

FIGS. 2-4 show sequences of biomarkers useful in the invention.

### DESCRIPTION OF EMBODIMENTS

The present invention provides compositions and methods for the detection or quantification of a set of particular biomarkers (including, but not limited to, ghrelin (e.g. total ghrelin), obestatin, cholecystokinin, glucagon-like peptide 1 (GLP-1, e.g. GLP-1(6-37)-NH₂), neuropeptide Y (NPY) and proopiomelanocortin (e.g. α-melanocyte stimulating hormone (α-MSH)), as defined herein) that allow for determining appetite regulation in a subject. The panels of biomarkers disclosed herein provide insight into the consequences of therapeutic intervention on hormonal satiety mechanisms. A number of drugs for the treatment of obesity have been developed and are available on the market. The biomarkers disclosed herein allow for determining a subject's level of response to drugs such as antiobesity drugs and for monitoring the effectiveness of drugs in a subject. Thus, measurement of the presence or quantity of the biomarkers provided herein allows for selection and monitoring of efficient risk-reducing treatment to avoid complications associated with treatments affecting appetite regulation.

A large number of biomarkers are known for a variety of metabolic, diabetic and cardiovascular conditions. See US/2008/0057590, incorporated by reference in its entirety. However, the present invention is particularly directed to the use of a minimum number of biomarkers to provide a maximum amount of information concerning appetite regulation in a subject. It has been found that ghrelin (e.g. total ghrelin), obestatin, cholecystokinin, GLP-1 (e.g. GLP-1(6-37)-NH₂), NPY and proopiomelanocortin (e.g., α-MSH) in combination are useful as biomarkers for appetite regulation, partly because, as discussed below, each allows the assessment of a different aspect of appetite regulation. A panel of biomarkers comprising or consisting of ghrelin (e.g. total ghrelin), obestatin, cholecystokinin, GLP-1 (e.g. GLP-1(6-37)-NH₂), NPY and proopiomelanocortin (e.g., α-MSH) may be combined with measurements of other biomarkers and clinical parameters to assess appetite regulation.

Thus, the invention provides biological markers that in various combinations can be used in methods to monitor subjects that are undergoing therapies affecting appetite regulation. Indications of appetite regulation allow a caregiver to select or modify therapies or interventions for treating subjects.

### Biomarkers

Biomarkers may originate from epidemiological studies, animal studies, pathophysiological considerations and end-organ experiments. Ideally, a biomarker will have a high predictive value for a meaningful outcome measure, can be or is validated in appropriately designed prospective trials, reflects therapeutic success by corresponding changes in the surrogate marker results, and should be easy to assess in clinical practice.

The term “surrogate marker,” “biomolecular marker,” “biomarker” or “marker” (also sometimes referred to herein as a “target analyte,” “target species” or “target sequence”) refers to a molecule whose measurement provides information as to the state of a subject. In various exemplary embodiments, the biomarker is used to assess a pathological state. Measurements of the biomarker may be used alone or combined with other data obtained regarding a subject in order to determine the state of the subject. In one embodiment, the biomarker is “differentially present” in a sample taken from a subject of one phenotypic status (e.g., having a disease) as compared with another phenotypic status (e.g., not having the disease). In one embodiment, the biomarker is “differentially present” in a sample taken from a subject undergoing no therapy or one type of therapy as compared with another type of therapy. Alternatively, the biomarker may be “differentially present” even if there is no phenotypic difference, e.g. the biomarkers may allow the detection of asymptomatic risk. A biomarker may be determined to be “differentially present” in a variety of ways, for example, between different phenotypic statuses if the mean or median level or concentration (particularly the expression level of the associated mRNAs as described below) of the biomarker in the different groups is calculated to be statistically significant. Common tests for statistical significance include, among others, t-test, ANOVA, Kruskal-Wallis, Wilcoxon, Mann-Whitney and odds ratio.

As described herein, a biomarker may be, for example, a small molecule, an analyte or target analyte, a lipid (including glycolipids), a carbohydrate, a nucleic acid, a protein, any derivative thereof or a combination of these molecules, with proteins and nucleic acids finding particular use in the invention. As will be appreciated by those in the art, a large number of analytes may be detected using the present methods; basically, any biomarker for which a binding ligand, described below, may be made may be detected using the methods of the invention.

In various embodiments, the biomarkers used in the panels of the invention can be detected either as proteins (i.e., polypeptides) or as nucleic acids (e.g. mRNA or cDNA transcripts) in any combination. In various embodiments, the protein form of a biomarker is measured. As will be appreciated by those in the art, protein assays may be done using standard techniques such as ELISA assays. In various embodiments, the nucleic acid form of a biomarker (e.g., the corresponding mRNA) is measured. In various exemplary embodiments, one or more biomarkers from a particular panel are measured using a protein assay and one or more biomarkers from the same panel are measured using a nucleic acid assay.

As will be appreciated by those in the art, there are a large number of possible proteinaceous target analytes and target species that may be detected using the present invention. The term “protein,” “polypeptide” or “oligopeptide” (used interchangeably herein) refers to at least two or more peptides or amino acids joined by one or more peptide bonds. A protein or an amino acid may be naturally or normaturally occurring and may be also be an analog, a derivative or a peptidomimetic structure. A protein can have a wild-type sequence, a variant of a wild-type sequence or either of these containing one or more analogs or derivatized amino acids. A variant may contain one or more additions, deletions or substitutions of one or more peptides compared to wild-type or a different variant sequence. Examples of derivatized amino acids include, without limitation, those that have been modified by the attachment of labels (described below); acetylation; acylation; ADP-ribosylation; amidation; covalent attachment of flavin, a heme moiety, a nucleotide, a lipid or phosphatidylinositol; cross-linking; cyclization; disulfide bond formation; demethylation; esterification; formation of covalent crosslinks, cystine or pyroglutamate; formylation; gamma carboxylation; glycosylation; GPI anchor formation; hydroxylation; iodination; methylation; myristoylation; oxidation; proteolytic processing; phosphorylation; prenylation; racemization; selenoylation; sulfation; and ubiquitination. Such modifications are well-known to those of skill in the art and have been described in great detail in the scientific literature. Several particularly common modifications such as glycosylation, lipid attachment, sulfation, gamma-carboxylation, hydroxylation and ADP-ribosylation, for instance, are described in basic texts, such as Creighton, Proteins—Structure and Molecular Properties, 2d ed. (New York: W.H. Freeman and Company, 1993). Many detailed reviews are available on this subject, such as in Johnson, ed., Posttranslational Covalent Modification of Proteins (New York: Academic Press, 1983); Seifter et al., Meth. Enzymol., 1990, 182: 626-646; and Rattan et al., Ann. N.Y. Acad. Sci., 1992, 663: 48-62. As discussed below, when the protein is used as a binding ligand, it may be desirable to utilize protein analogs to retard degradation by sample contaminants.

In various exemplary embodiments, the biomarker is a nucleic acid. The term “nucleic acid,” “oligonucleotide” or “polynucleotide” herein means at least two nucleotides covalently linked together. A nucleic acid of the present invention will generally contain phosphodiester bonds, although in some cases, for example in the use of binding ligand probes, nucleic acid analogs are included that may have alternate backbones, comprising, for example, phosphoramide (Beaucage et al., Tetrahedron, 49(10): 1925 (1993) and references therein; Letsinger, J. Org. Chem. 35: 3800 (1970); Sprinzl et al., Eur. J. Biochem. 81:579 (1977); Letsinger et al., Nucl. Acids Res. 14: 3487 (1986); Sawai et al, Chem. Lett. 13(5): 805 (1984); Letsinger et al., J. Am. Chem. Soc. 110:4470 (1988); and Pauwels et al., Chemica Scripta 26:141 (1986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, (Oxford University Press, 1991), and peptide nucleic acid backbones and linkages (see Egholm, J. Am. Chem. Soc. 114: 1895 (1992); Meier et al., Chem. Int. Ed. Engl. 31: 1008 (1992); Nielsen, Nature, 365: 566 (1993); Carlsson et al., Nature, 380: 207 (1996), all of which are incorporated by reference). Other analog nucleic acids include those with positive backbones (Denpcy et al., Proc. Natl. Acad. Sci. USA 92: 6097 (1995)), non-ionic backbones (U.S. Pat. Nos. 5,386,023; 5,637,684; 5,602,240; 5,216,141 and 4,469,863; Kiedrowshi et al., Angew. Chem. Intl. Ed. English 30: 423 (1991); Letsinger et al., J. Am. Chem. Soc. 110: 4470 (1988); Letsinger et al., Nucleoside & Nucleotide 13: 1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4: 395 (1994); Jeffs et al., J. Biomolecular NMR 34: 17 (1994); and Horn et al., Tetrahedron Lett. 37: 743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev., 24: 169-176 (1995)). Several nucleic acid analogs are described in Rawls, C & E News, 35 (Jun. 2, 1997). All of these references are hereby expressly incorporated by reference. These modifications of the ribose-phosphate backbone may be done to increase the stability and half-life of such molecules in physiological environments. As will be appreciated by those in the art, all of these nucleic acid analogs may find use in the present invention. In addition, mixtures of naturally occurring nucleic acids and analogs can be made.

In various embodiments, variants of the sequences described herein, including proteins and nucleic acids based on e.g. splice variants, variants comprising a deletion, addition, substitution, fragment, preproprotein, processed preproprotein (e.g. without a signaling peptide), processed proprotein (e.g. resulting in an active form), nonhuman sequences and variant nonhuman sequences may be used as biomarkers. In some embodiments, the variant sequence has a homology compared to a parent sequence, such as a sequence described herein, of about a percentage selected from 30%, 40%, 50%, 60%, 70%, 80%, 90%, 91%, 92%, 93%, 94%, 95%, 96%, 97%, 98% and 99%.

It has been found that assays for appetite regulation involving the measurement of ghrelin (e.g. total ghrelin), obestatin, cholecystokinin, GLP-1 (e.g. GLP-1(6-37)-NH₂), NPY and proopiomelanocortin (e.g., α-MSH) have greater value in determining appetite regulation than those involving any of these biomarkers alone. This particular combination, as well as other combinations of these biomarkers, allows attainment of clinically useful sensitivity and specificity. Accordingly, measurements of a biomarker panel comprising or consisting of ghrelin, obestatin, cholecystokinin, GLP-1, NPY and proopiomelanocortin in various combinations may be used to improve the sensitivity and/or specificity of a diagnostic test compared to a test involving any one of these biomarkers alone.

### Ghrelin

In various embodiments, ghrelin is used as a biomarker. Ghrelin is the natural ligand of GH secretagogue receptor 1a (GHS-R1a) and is an orexigenic peptide whose levels increase during fasting. Ghrelin is derived from preproghrelin (also known as ghrelin or obestatin preproprotein), a 117 amino acid precursor (see, for example, RefSeq Accession Record NP_{—}057446). Preproghrelin consists of a 23 amino acid signal polypeptide and a 94 amino acid polypeptide called proghrelin. Proghrelin is subsequently cleaved and acylated, resulting in the ghrelin polypeptide. In its natural major active form, ghrelin has 28 amino acids and is esterified with an octanoic acid on the hydroxyl group of serine at position 3 (S3). Other forms of the peptide that have been observed include those in which S3 is nonacylated, decanoylated or decenoylated. Still other forms of ghrelin have 27 amino acids that are possibly derivatized. Thus, as used herein, ghrelin can refer to a 27 amino acid (“ghrelin (1-27)”) or 28 amino acid (“ghrelin (1-28)”) fragment of preproghrelin, which fragment can be derivatized (e.g., acylated) or underivatized. In exemplary embodiments, ghrelin refers to total ghrelin, which is the sum of both acylated and deacylated (or desacyl) forms.

In various embodiments, ghrelin has 27 or 28 amino acids and is optionally derivatized. In exemplary embodiments, ghrelin has the sequence GSSFLSPEHQRVQQRKESKKPPAKLQPR (SEQ ID NO: 1) wherein S3 is optionally acylated and in particular wherein S3 is optionally octanoylated. In exemplary embodiments, ghrelin has the sequence GSSFLSPEHQRVQQRKESKKPPAKLQP (SEQ ID NO: 2) wherein S3 is optionally acylated and in particular wherein S3 is optionally octanoylated. In various embodiments, ghrelin is selected from decanoyl ghrelin (1-28), decenoyl ghrelin (1-28), octanoyl ghrelin (1-27) and decanoyl ghrelin (1-27).

In various embodiments, any derivatized (e.g. acylated) or underivatized fragment of preproghrelin or derivatized (e.g. acylated) or underivatized full-length preproghrelin (for example according to RefSeq Accession Record NP_{—}057446) is used as a biomarker. In various embodiments, any derivatized or underivatized fragment of proghrelin is used as a biomarker. For example, in various embodiments, a 66 amino acid portion of wild type proghrelin (29-94) known as C-ghrelin can be measured.

In an exemplary embodiment, a polypeptide form of ghrelin is measured. Accordingly, suitable capture binding ligands, as further discussed below, for detection and/or quantification of ghrelin include, but are not limited to, antibodies that are selective for ghrelin.

In one embodiment, a nucleic acid form of ghrelin (e.g. mRNA derived from a sequence according to RefSeq Accession Record NM_{—}016362 or a fragment thereof) is measured. A wide variety of methods for detecting mRNA are known in the art, particularly on arrays. This includes the direct measurement of mRNA as well as treating the same with reverse transcriptase and measuring cDNA levels. Accordingly, suitable capture probes, as further discussed below, for the detection and/or quantification of ghrelin mRNA include, but are not limited to, fragments of the complements of the mRNA sequences of ghrelin. That is, if the mRNA is to be directly detected, a complementary sequence will be used to bind the single stranded mRNA. In general, as for all the capture probes outlined herein, the probes generally are between about 5 and about 100 nucleotides in length, with from about 6 to about 30, about 8 to about 28, and about 16 to about 26 being of particular use in some embodiments.

In response to a therapy, such as administration of a disease-modulating drug, as described below, the levels of ghrelin (e.g. total ghrelin) will decrease if the patient is responding to the therapy. In some embodiments, this decrease is about 10% to about 80%, about 20% to about 70%, about 30% to about 60% or about 40% to about 50% from a reference value. In some embodiments, this decrease is about 10% to about 20%, about 10% to about 30%, about 10% to about 40%, about 10% to about 50%, about 10% to about 60%, about 10% to about 70%, about 10% to about 80%, or about 10% to about 90% from a reference value. In some embodiments, a decrease of at least about a percentage selected from 10%, 11%, 12%, 13%, 14%, 15%, 16%, 17%, 18%, 19%, 20%, 30%, 40%, 50%, 60%, 70%, 80%, 90%, 95%, 96%, 97%, 98% and 99% from a reference value will occur. In exemplary embodiments, a decrease of about 10% to about 40% from a reference value occurs. In exemplary embodiments, a decrease of at least about 15% from a reference value occurs. In exemplary embodiments, plasma or serum levels of ghrelin decrease to below a level selected from about 0.5 μg/L, about 0.6 μg/L, about 0.7 μg/L, about 0.8 μg/L, about 0.9 μg/L, about 1 μg/L, about 2 μg/L, about 3 μg/L, about 4 μg/L, about 5 μg/L, about 6 μg/L or about 7 μg/L, preferably about 5 μg/L. In various embodiments, a response to a therapy will cause ghrelin to change from an initial level to a level either above or below a reference value, such as a level selected from about 0.5 μg/L, about 0.6 μg/L, about 0.7 μg/L, about 0.8 μg/L, about 0.9 μg/L, about 1 μg/L, about 2 μg/L, about 3 μg/L, about 4 μg/L, about 5 μg/L, about 6 μg/L or about 7 μg/L, preferably about 5 μg/L. A reference range for ghrelin or total ghrelin is <5 μg/L, with elevated levels at >5 μg/L.

As is more fully described below, it is also possible that the patient is responding to a therapy, such as administration of a disease-modulating drug, as shown by changes in other biomarkers, but the levels of ghrelin are not changing in a significant way.

### Obestatin

In various embodiments, obestatin is used as a biomarker. In contrast to ghrelin, obestatin is thought to suppress food intake, inhibit jejunal contraction and decrease body-weight gain. Zhang et al., Science, 2005, 310: 996-999 and Supplemental Online Material (DOI: 10.1126/science.1117255; accessible at http://www.sciencemag.org/cgi/data/310/5750/996/DC1/1). Obestatin is a polypeptide derived from proghrelin, such as described above. In exemplary embodiments, obestatin has the sequence FNAPFDVGIKLSGVQYQQHSQAL (SEQ ID NO: 3). In various embodiments, obestatin is underivatized or derivatized, and in various embodiments, obestatin is amidated.

In an exemplary embodiment, a polypeptide form of obestatin is measured. Accordingly, suitable capture binding ligands, as further discussed below, for detection and/or quantification of obestatin include, but are not limited to, antibodies that are selective for obestatin.

In one embodiment, a nucleic acid form of obestatin (e.g. mRNA derived from ghrelin mRNA as described above) is measured. A wide variety of methods for detecting mRNA are known in the art, particularly on arrays. This includes the direct measurement of mRNA as well as treating the same with reverse transcriptase and measuring cDNA levels. Accordingly, suitable capture probes, as further discussed below, for the detection and/or quantification of obestatin mRNA include, but are not limited to, fragments of the complements of the mRNA sequences of obestatin. That is, if the mRNA is to be directly detected, a complementary sequence will be used to bind the single stranded mRNA. In general, as for all the capture probes outlined herein, the probes generally are between about 5 and about 100 nucleotides in length, with from about 6 to about 30, about 8 to about 28, and about 16 to about 26 being of particular use in some embodiments.

In response to a therapy, such as administration of a disease-modulating drug, as described below, the levels of obestatin will increase if the patient is responding to the therapy. In some embodiments, this increase is about 10% to about 80%, about 20% to about 70%, about 30% to about 60% or about 40% to about 50% from a reference value. In some embodiments, this increase is about 10% to about 20%, about 10% to about 30%, about 10% to about 40%, about 10% to about 50%, about 10% to about 60%, about 10% to about 70%, about 10% to about 80% or about 10% to about 90% from a reference value. In some embodiments, this increase is about 50% to about 100%, about 50% to about 110%, about 50% to about 120%, about 50% to about 130%, about 50% to about 140% or about 50% to about 150% from a reference value. In some embodiments, an increase of at least about a percentage selected from 10%, 20%, 30%, 40%, 50%, 60%, 70%, 80%, 90%, 100%, 110%, 120%, 130%, 140%, 150%, 160%, 170%, 180%, 190% and 200% from a reference value occurs. In exemplary embodiments, an increase of about 10% to about 40% from a reference value occurs. In exemplary embodiments, an increase of at least about 15% from a reference value occurs. In some embodiments, plasma or serum levels of obestatin increase from an initial level to above a level selected from about 20 ng/L, about 30 ng/L, about 40 ng/L, about 50 ng/L, about 60 ng/L, about 100 ng/L, about 150 ng/L, about 200 ng/L and about 250 ng/L, preferably above about 40 ng/L. In exemplary embodiments, a response to a therapy will cause obestatin to change from an initial level to a level either above or below a reference value, such as a level selected from 20 ng/L, about 30 ng/L, about 40 ng/L, about 50 ng/L, about 60 ng/L, about 100 ng/L, about 150 ng/L, about 200 ng/L and about 250 ng/L, preferably about 40 ng/L. A reference range for obestatin is >40 ng/L, with obesity levels at <40 ng/L.

As is more fully described below, it is also possible that the patient is responding to a therapy, such as administration of a disease-modulating drug, as shown by changes in other biomarkers, but the levels of obestatin are not changing in a significant way.

### Cholecystokinin

In various embodiments, cholecystokinin (CCK) is used as a biomarker. Cholecystokinin is found in the brain and the gut. In the gut, it induces the release of pancreatic enzymes and the contraction of the gallbladder while in the brain, its physiologic role is unclear. CCK is a peptide known to be important for satiety, and when administered exogenously has been shown to reduce food intake. The cholecystokinin prohormone is processed by endo- and exoproteolytic cleavages. Thus, cholecystokinin as used herein refers to preprocholecystokinin (for example, according to RefSeq Accession Record NP_{—}000720) or preferably a fragment thereof, any of which can be derivatized or underivatized. Cleavage by endopeptidases after a single basic residue results in CCK-58, which is a large biologically active form of cholecystokinin isolated from intestine and brain tissue. Eberlein et al., Journal of Biological Chemistry, 1992, 267: 1517-1521. CCK-58 is the major stored and circulating form of cholecystokinin in humans. Smaller forms of cholecystokinin, namely CCK-8, CCK-22, CCK-33 and CCK-39, result from cleavage by endopeptidases after other single basic residues. Many other forms are known in the art and may be used herein. Thus, in various embodiments, cholecystokinin refers to, for example, CCK-4, CCK-8, CCK-12, CCK-22, CCK-33, CCK-39, CCK-58, any CCK fragment from cleavage after double basic residues (e.g., CCK-61) or CCK-83. In various embodiments, cholecystokinin has the sequence

(SEQ ID NO: 4)
VSQRTDGESR AHLGALLARY IQQARKAPSG RMSIVKNLQN
LDPSHRISDR DYMGWMDF

wherein the tyrosine that is seven positions from the carboxyl terminus is optionally sulfated.

Preprocholescystokinin is modified by several types of processing to form the biologically active peptide. Thus, in various embodiments, cholecystokinin refers to a derivatized or underivatized fragment of preprocholescystokinin (for example, according to RefSeq Accession Record NP_{—}000720). In various exemplary embodiments, cholecystokinin comprises an amidated phenylalanine at the carboxyl terminus and a sulfated tyrosine six residues before the carboxyl terminus.

In an exemplary embodiment, a polypeptide form of cholecystokinin is measured. Accordingly, suitable capture binding ligands, as further discussed below, for detection and/or quantification of cholecystokinin include, but are not limited to, antibodies that are selective for cholecystokinin.

In one embodiment, a nucleic acid form of cholecystokinin (e.g. mRNA derived from a sequence according to RefSeq Accession Record NM_{—}000729) is measured. A wide variety of methods for detecting mRNA are known in the art, particularly on arrays. This includes the direct measurement of mRNA as well as treating the same with reverse transcriptase and measuring cDNA levels. Accordingly, suitable capture probes, as further discussed below, for the detection and/or quantification of cholecystokinin mRNA include, but are not limited to, fragments of the complements of the mRNA sequences of cholecystokinin. That is, if the mRNA is to be directly detected, a complementary sequence will be used to bind the single stranded mRNA. In general, as for all the capture probes outlined herein, the probes generally are between about 5 and about 100 nucleotides in length, with from about 6 to about 30, about 8 to about 28, and about 16 to about 26 being of particular use in some embodiments.

In response to a therapy, such as administration of a disease-modulating drug, as described below, the levels of cholecystokinin will increase if the patient is responding to the therapy. In some embodiments, this increase is about 10% to about 80%, about 20% to about 70%, about 30% to about 60% or about 40% to about 50% from a reference value. In some embodiments, this increase is about 10% to about 20%, about 10% to about 30%, about 10% to about 40%, about 10% to about 50%, about 10% to about 60%, about 10% to about 70%, about 10% to about 80% or about 10% to about 90% from a reference value. In some embodiments, this increase is about 50% to about 100%, about 50% to about 110%, about 50% to about 120%, about 50% to about 130%, about 50% to about 140% or about 50% to about 150% from a reference value. In some embodiments, an increase of at least about a percentage selected from 10%, 20%, 30%, 40%, 50%, 60%, 70%, 80%, 90%, 100%, 110%, 120%, 130%, 140%, 150%, 160%, 170%, 180%, 190% and 200% from a reference value occurs. In exemplary embodiments, an increase of about 10% to about 40% from a reference value occurs. In exemplary embodiments, an increase of at least about 15% from a reference value occurs. In some embodiments, plasma or serum levels of cholecystokinin increase from an initial level to above a level selected from about 0.5 μg/L, about 0.6 μg/L, about 0.7 μg/L, about 0.8 μg/L, about 0.9 μg/L, about 1.0 μg/L, about 1.1 μg/L and about 1.2 μg/L, preferably above about 1.0 μg/L. In exemplary embodiments, a response to a therapy will cause cholecystokinin to change from an initial level to a level either above or below a reference value, such as a level selected from about 0.5 μg/L, about 0.6 μg/L, about 0.7 μg/L, about 0.8 μg/L, about 0.9 μg/L, about 1.0 μg/L, about 1.1 μg/L and about 1.2 μg/L, preferably about 1.0 μg/L. A reference range for cholecystokinin is <1 μg/L, with elevated levels at >1 μg/L.

As is more fully described below, it is also possible that the patient is responding to a therapy, such as administration of a disease-modulating drug, as shown by changes in other biomarkers, but the levels of cholecystokinin are not changing in a significant way.

glucagon-Like Peptide 1 (GLP-1)

In various embodiments, glucagon-like peptide 1 (GLP-1) is used as a biomarker. GLP-1 is an incretin released from L-cells in the small intestine. Peripheral administration of GLP-1 has been shown to decrease food intake. GLP-1 is also a fragment of proglucagon, which itself is a 160 amino acid fragment of preproglucagon. See Holst et al., Physiological Reviews, 2007, 87: 1409-1439. The predominant biologically active form of GLP-1 in humans is GLP-1(7-36)-NH₂, which corresponds to proglucagon (78-107). GLP-1(7-36)-NH₂ is rapidly inactivated in the body. For example, dipeptidyl peptidase IV (DPP IV) degrades GLP-1(7-36)-NH₂ to GLP-1(9-36)-NH₂. Enzymes such as neutral endopeptidase (NEP) 24.11 are known to hydrolyze GLP-1(7-36)-NH₂ at positions 15, 16, 18, 19, 20, 27, 28, 31 and 32. Thus, in various embodiments, GLP-1 refers to preproglucagon (for example, according to RefSeq Accession Record NP_{—}002045 or the nucleic acid sequence in RefSeq Accession Record NM_{—}002054) or preferably a fragment thereof, any of which can be derivatized or underivatized. In various embodiments, GLP-1 refers to a derivatized or underivatized fragment of proglucagon. In various exemplary embodiments, GLP-1 refers to, for example, GLP-1(7-36)-NH₂, GLP-1(7-37), GLP-1(9-36)-NH₂, or any fragment resulting from the hydrolysis of GLP-1(7-36)-NH₂ at positions 15, 16, 18, 19, 20, 27, 28, 31, 32 or other positions. In various embodiments, GLP-1 has the sequence HAEGTFTSDVSSYLEGQAAKEFIAWLVKGR (SEQ ID NO: 5), wherein the final arginine is optionally amidated. In exemplary embodiments, GLP-1 refers to GLP-1(6-37), which is preferably amidated. Other GLP-1 polypeptides useful in the present invention can be found in Chi et al., Bioorganic and Medicinal Chemistry, 2008, 16: 7607-7614, Holst et al., Physiological Reviews, 2007, 87: 1409-1439 (all incorporated by reference in their entirety for all purposes) and in other references in the art.

In various embodiments, GLP-1 refers to an amidated fragment of preproglucagon or an amidated fragment of proglucagon.

In an exemplary embodiment, a polypeptide form of GLP-1 is measured. Accordingly, suitable capture binding ligands, as further discussed below, for detection and/or quantification of GLP-1 include, but are not limited to, antibodies that are selective for GLP-1.

In one embodiment, a nucleic acid form of GLP-1 (e.g. mRNA derived from a sequence according to RefSeq Accession Record NM_{—}002054) is measured. A wide variety of methods for detecting mRNA are known in the art, particularly on arrays. This includes the direct measurement of mRNA as well as treating the same with reverse transcriptase and measuring cDNA levels. Accordingly, suitable capture probes, as further discussed below, for the detection and/or quantification of GLP-1 mRNA include, but are not limited to, fragments of the complements of the mRNA sequences of GLP-1. That is, if the mRNA is to be directly detected, a complementary sequence will be used to bind the single stranded mRNA. In general, as for all the capture probes outlined herein, the probes generally are between about 5 and about 100 nucleotides in length, with from about 6 to about 30, about 8 to about 28, and about 16 to about 26 being of particular use in some embodiments.

In response to a therapy, such as administration of a disease-modulating drug, as described below, the levels of GLP-1 (e.g. GLP-1(6-37)-NH₂) will increase if the patient is responding to the therapy. In some embodiments, this increase is about 10% to about 80%, about 20% to about 70%, about 30% to about 60% or about 40% to about 50% from a reference value. In some embodiments, this increase is about 10% to about 20%, about 10% to about 30%, about 10% to about 40%, about 10% to about 50%, about 10% to about 60%, about 10% to about 70%, about 10% to about 80% or about 10% to about 90% from a reference value. In some embodiments, this increase is about 50% to about 100%, about 50% to about 110%, about 50% to about 120%, about 50% to about 130%, about 50% to about 140% or about 50% to about 150% from a reference value. In some embodiments, an increase of at least about a percentage selected from 10%, 20%, 30%, 40%, 50%, 60%, 70%, 80%, 90%, 100%, 110%, 120%, 130%, 140%, 150%, 160%, 170%, 180%, 190% and 200% from a reference value occurs. In exemplary embodiments, an increase of about 10% to about 40% from a reference value occurs. In exemplary embodiments, an increase of at least about 15% from a reference value occurs. In some embodiments, plasma or serum levels of GLP-1 increase from an initial level to above a level selected from about 15 pg/mL, about 20 pg/mL, about 25 pg/mL, about 30 pg/mL and about 35 pg/mL, preferably above about 20 pg/mL. In exemplary embodiments, a response to a therapy will cause GLP-1 to change from an initial level to a level either above or below a reference value, such as a level selected from about 15 pg/mL, about 20 pg/mL, about 25 pg/mL, about 30 pg/mL and about 35 pg/mL, preferably about 20 pg/mL. A reference range for GLP-1 such as GLP-1(6-37)-NH₂ is <20 pg/mL, with elevated levels at >20 pg/mL.

As is more fully described below, it is also possible that the patient is responding to a therapy, such as administration of a disease-modulating drug, as shown by changes in other biomarkers, but the levels of GLP-1 are not changing in a significant way.

### Neuropeptide Y (NPY)

In various embodiments, neuropeptide Y (NPY) is used as a biomarker. NPY is a neurotransmitter found throughout the central and peripheral nervous system. NPY has been found to stimulate food intake and inhibit release of luteinizing hormone. NPY is also known to cause the increased storage of ingested food as fat. In various exemplary embodiments, NPY refers to NPY preproprotein (for example, according to RefSeq Accession Record NP_{—}000896) or preferably a fragment thereof, any of which can be derivatized or underivatized. In various exemplary embodiments, NPY has the sequence YPSKPDNPGEDAPAEDMARYYSALRHYINLITRQRY (SEQ ID NO: 6). Sequences of NPY that are useful in the present invention can be found, for example, in Minth et al., Journal of Biological Chemistry, 1986, 261(26): 11974-11979; and Darbon et al., European Journal of Biochemistry, 1992, 209: 765-771, all incorporated by reference in their entirety for all purposes.

In an exemplary embodiment, a polypeptide form of NPY is measured. Accordingly, suitable capture binding ligands, as further discussed below, for detection and/or quantification of NPY include, but are not limited to, antibodies that are selective for NPY.

In one embodiment, a nucleic acid form of NPY (e.g. mRNA derived from a sequence according to RefSeq Accession Record NM_{—}000905) is measured. A wide variety of methods for detecting mRNA are known in the art, particularly on arrays. This includes the direct measurement of mRNA as well as treating the same with reverse transcriptase and measuring cDNA levels. Accordingly, suitable capture probes, as further discussed below, for the detection and/or quantification of NPY mRNA include, but are not limited to, fragments of the complements of the mRNA sequences of NPY. That is, if the mRNA is to be directly detected, a complementary sequence will be used to bind the single stranded mRNA. In general, as for all the capture probes outlined herein, the probes generally are between about 5 and about 100 nucleotides in length, with from about 6 to about 30, about 8 to about 28, and about 16 to about 26 being of particular use in some embodiments.

In response to a therapy, such as administration of a disease-modulating drug, as described below, the levels of NPY will decrease if the patient is responding to the therapy. In some embodiments, this decrease is about 10% to about 80%, about 20% to about 70%, about 30% to about 60% or about 40% to about 50% from a reference value. In some embodiments, this decrease is about 10% to about 20%, about 10% to about 30%, about 10% to about 40%, about 10% to about 50%, about 10% to about 60%, about 10% to about 70%, about 10% to about 80%, or about 10% to about 90% from a reference value. In some embodiments, a decrease of at least about a percentage selected from 10%, 11%, 12%, 13%, 14%, 15%, 16%, 17%, 18%, 19%, 20%, 30%, 40%, 50%, 60%, 70%, 80%, 90%, 95%, 96%, 97%, 98% and 99% from a reference value will occur. In exemplary embodiments, a decrease of about 10% to about 40% from a reference value occurs. In exemplary embodiments, a decrease of at least about 15% from a reference value occurs. In some embodiments, plasma or serum levels of NPY decrease from an initial level to below a level selected from about 8 pmol/L, about 9 pmol/L, about 10 pmol/L, about 11 pmol/L, about 12 pmol/L, about 13 pmol/L, about 14 pmol/L and about 15 pmol/L. In exemplary embodiments, plasma or serum levels of NPY decrease from an initial level to about 10-13 pmol/L. In exemplary embodiments, plasma or serum levels of NPY decrease from an initial level to below about 10 pmol/L. In exemplary embodiments, a response to a therapy will cause NPY to change from an initial level to a level either above or below a reference value, such as a level selected from 8 pmol/L, about 9 pmol/L, about 10 pmol/L, about 11 pmol/L, about 12 pmol/L, about 13 pmol/L, about 14 pmol/L and about 15 pmol/L. A reference range for NPY is about 10-13 pmol/L, with hunger levels at >13 pmol/L and satiety levels at <10 pmol/L.

As is more fully described below, it is also possible that the patient is responding to a therapy, such as administration of a disease-modulating drug, as shown by changes in other biomarkers, but the levels of NPY are not changing in a significant way.

### Proopiomelanocortin (POMC)

In various embodiments, proopiomelanocortin (POMC) is used as a biomarker. Proopiomelanocortin is a polypeptide hormone precursor that undergoes tissue-specific, posttranslational processing via cleavage by enzymes such as the prohormone convertases. Adrenocorticotropin, which is essential for normal steroidogenesis and the maintenance of normal adrenal weight, and lipotropin beta are the major end products. In tissues such as the hypothalamus, placenta, and epithelium, cleavage of proopiomelanocortin results in peptides having a role in pain and energy homeostasis, melanocyte stimulation and immune modulation. These include several distinct melanotropins, lipotropins and endorphins. Thus, as used herein, the term “proopiomelanocortin” refers to the full length polypeptide hormone precursor, e.g. as provided by RefSeq Accession Record NP_{—}000930 or preferably a fragment thereof, any of which can be derivatized or underivatized. In various embodiments, proopiomelanocortin refers to adrenocorticotropic hormone (ACTH, corticotropin or adrenocorticotropin), proATCH, joining peptide, N-terminal proopiocortin (N-POC), ACTH(1-17), desacetyl-αMSH (da-αMSH), α-melanocyte stimulating hormone (α-MSH), β-melanocyte stimulating hormone (β-MSH), γ-melanocyte stimulating hormone (γ-MSH), β-lipotropin, γ-lipotropin, corticotropin-like intermediate peptide (CLIP), β-endorphin or met-enkephalin. In various embodiments, proopiomelanocortin refers to a sequence selected from Table 1. Sequences of proopiomelanocortin useful in the present invention can be found in, for example, Pritchard & White, Endocrinology, 2007, 148(9): 4201-4207; Nillni, Endocrinology, 2007, 148(9): 4191-4200; and Raffin-Sanson et al., European Journal of Endocrinology, 2003, 149(2): 79-90, all incorporated by reference in their entirety for all purposes.

TABLE 1

| | Exemplary Fragment of Full- |
|---|---|
| | Length Proopiomelanocortin |
| | Sequence According to RefSeq |
| Polypeptide | Accession Record NP_000930 |
| proprotein | 27-267 |
| N-terminal peptide | 27-102 |
| melanotropin gamma | 77-87 |
| joining peptide | 105-134 |
| adrenocorticotropin | 138-176 |
| melanotropin alpha | 138-150 |
| corticotropin-like | 156-176 |
| intermediary peptide | |
| lipotropin beta | 179-267 |
| lipotropin gamma | 179-234 |
| melanotropin beta | 217-234 |
| beta-endorphin | 237-267 |
| beta-endorphin (1-27) | 237-263 |
| met-enkephalin | 237-241 |

In an exemplary embodiment, proopiomelanocortin refers to α-MSH, having the sequence Ac-SYSMEHFRWGKPV (SEQ ID NO: 7). α-MSH is a known inhibitor of appetite.

In an exemplary embodiment, a polypeptide form of proopiomelanocortin is measured. Accordingly, suitable capture binding ligands, as further discussed below, for detection and/or quantification of proopiomelanocortin include, but are not limited to, antibodies that are selective for proopiomelanocortin.

In one embodiment, a nucleic acid form of proopiomelanocortin (e.g. mRNA derived from a sequence according to RefSeq Accession Record NM_{—}000939) is measured. A wide variety of methods for detecting mRNA are known in the art, particularly on arrays. This includes the direct measurement of mRNA as well as treating the same with reverse transcriptase and measuring cDNA levels. Accordingly, suitable capture probes, as further discussed below, for the detection and/or quantification of proopiomelanocortin mRNA include, but are not limited to, fragments of the complements of the mRNA sequences of proopiomelanocortin. That is, if the mRNA is to be directly detected, a complementary sequence will be used to bind the single stranded mRNA. In general, as for all the capture probes outlined herein, the probes generally are between about 5 and about 100 nucleotides in length, with from about 6 to about 30, about 8 to about 28, and about 16 to about 26 being of particular use in some embodiments.

In response to a therapy, such as administration of a disease-modulating drug, as described below, the levels of proopiomelanocortin (e.g., α-MSH) will increase if the patient is responding to the therapy. In some embodiments, this increase is about 10% to about 80%, about 20% to about 70%, about 30% to about 60% or about 40% to about 50% from a reference value. In some embodiments, this increase is about 10% to about 20%, about 10% to about 30%, about 10% to about 40%, about 10% to about 50%, about 10% to about 60%, about 10% to about 70%, about 10% to about 80% or about 10% to about 90% from a reference value. In some embodiments, this increase is about 50% to about 100%, about 50% to about 110%, about 50% to about 120%, about 50% to about 130%, about 50% to about 140% or about 50% to about 150% from a reference value. In some embodiments, an increase of at least about a percentage selected from 10%, 20%, 30%, 40%, 50%, 60%, 70%, 80%, 90%, 100%, 110%, 120%, 130%, 140%, 150%, 160%, 170%, 180%, 190% and 200% from a reference value occurs. In exemplary embodiments, an increase of about 10% to about 40% from a reference value occurs. In exemplary embodiments, an increase of at least about 15% from a reference value occurs. In some embodiments, plasma or serum levels of proopiomelanocortin increase from an initial level to above a level selected from about 10 ng/L, about 15 ng/L, about 20 ng/L, about 25 ng/L and about 30 ng/L, preferably above about 20 ng/L. In exemplary embodiments, a response to a therapy will cause proopiomelanocortin to change from an initial level to a level either above or below a reference value, such as a level selected from about 10 ng/L, about 15 ng/L, about 20 ng/L, about 25 ng/L and about 30 ng/L, preferably about 20 ng/L. A reference range for a proopiomelanocortin such as α-MSH is <20 ng/L, with elevated levels at >20 ng/L.

As is more fully described below, it is also possible that the patient is responding to a therapy, such as administration of a disease-modulating drug, as shown by changes in other biomarkers, but the levels of proopiomelanocortin are not changing in a significant way.

### Biomarker Panels

Any combination of the biomarkers described herein can be used to assemble a biomarker panel, which is detected or measured as described herein. As is generally understood in the art, a combination may refer to an entire set or any subset (i.e. subcombination) thereof. According to context, the term combination may mean more than one but fewer than all. The term “biomarker panel,” “biomarker profile,” or “biomarker fingerprint” refers to a set of biomarkers. As used herein, these terms can also refer to any form of the biomarker that is measured. Thus, if ghrelin is part of a biomarker panel, then either a ghrelin polypeptide or a ghrelin mRNA, for example, could be considered to be part of the panel. While individual biomarkers are useful as diagnostics, it has been found that a combination of biomarkers can sometimes provide greater value in determining a particular status than single biomarkers alone. Specifically, the detection of a plurality of biomarkers in a sample can increase the sensitivity and/or specificity of the test. Thus, in various embodiments, a biomarker panel may include 2, 3, 4, 5, 6, 7, 8, 9, 10 or more types of biomarkers. In various exemplary embodiments, the biomarker panel consists of a minimum number of biomarkers to generate a maximum amount of information. Thus, in various embodiments, the biomarker panel consists of 2, 3, 4, 5, 6, 7, 8, 9 or 10 types of biomarkers. Where a biomarker panel “consists of” a set of biomarkers, no biomarkers other than those of the set are present.

The present invention provides a biomarker panel comprising or consisting of any combination of the biomarkers outlined herein.

In various exemplary embodiments, the biomarker panel comprises additional biomarkers. Such additional biomarkers may, for example, increase the specificity and/or sensitivity the test. For example, additional biomarkers may be those that are currently evaluated in the clinical laboratory and used in traditional global risk assessment algorithms, such as those from the San Antonio Heart Study, the Framingham Heart Study, and the National Cholesterol Education Program Expert Panel on Detection, Evaluation, and Treatment of High Blood Cholesterol in Adults (Adult Treatment Panel III), also known as NCEP/ATP III. Additional biomarkers suitable for biomarker panels include, without limitation and if not already selected, any combination of biomarkers selected from adiponectin, angiotensin II, complement factor 3, leptin, mRNAx, NFκB, IL-6, MMP-9, TNFα, NFκB, eNOS, PPARγ, MCP-1, PAI-1, ICAM/VCAM, E-selectin, P-selectin, von Willebrand factor, sCD40L, insulin, proinsulin, glucose, HbA1c, lipids such as free fatty acids, total cholesterol, triglycerides, VLDL, LDL, small dense LDL, oxidized LDL, resistin, HDL, NO, IκB-α, IκB-β, p105, RelA, MIF, inflammatory cytokines, molecules involved in signaling pathways, traditional laboratory risk factors and any biomarkers disclosed in US/2008/0057590. Glucose as used herein includes, without limitation, fasting glucose as well as glucose concentrations taken during and after the oral glucose tolerance test, such as 120 minute Glucose. Insulin as used herein includes, without limitation, fasting insulin and insulin concentrations taken during and after the oral glucose tolerance test, such as 120 minute Insulin. Traditional laboratory risk factors are also understood to encompass without limitation, fibrinogen, lipoprotein (a), c-reactive protein (including hsCRP), D-dimer, and homocysteine. It should be understood that in these embodiments, the biomarker panel can include any combination of biomarkers selected from ghrelin (e.g. total ghrelin), obestatin, cholecystokinin, GLP-1 (e.g. GLP-1(6-37)-NH₂), NPY, proopiomelanocortin (e.g., α-MSH) and the remainder of these markers.

A biomarker can also be a clinical parameter, although in some embodiments, the biomarker is not included in the definition of “biomarker”. The term “clinical parameter” refers to all non-sample or non-analyte biomarkers of subject health status or other characteristics, such as, without limitation, age, ethnicity, gender, diastolic blood pressure and systolic blood pressure, family history, height, weight, waist and hip circumference, body-mass index, as well as others such as Type I or Type II Diabetes Mellitus or Gestational Diabetes Mellitus (collectively referred to here as Diabetes), resting heart rate, homeostatic model assessment (HOMA), HOMA insulin resistance (HOMA-IR), intravenous glucose tolerance (SI(IVGT)), β-cell function, macrovascular function, microvascular function, atherogenic index, blood pressure, low-density lipoprotein/high-density lipoprotein ratio, intima-media thickness, and UKPDS risk score. Other clinical parameters are disclosed in US/2008/0057590.

In various exemplary embodiments, the biomarker panel comprises total ghrelin, obestatin, cholecystokinin, GLP-1(6-37)-NH₂, NPY and α-MSH. In various exemplary embodiments, the biomarker panel comprises any combination of total ghrelin, obestatin, cholecystokinin, GLP-1(6-37)-NH₂, NPY and α-MSH. In various exemplary embodiments, the biomarker panel consists of total ghrelin, obestatin, cholecystokinin, GLP-1(6-37)-NH₂, NPY and α-MSH. In various exemplary embodiments, the biomarker panel consists of any combination of total ghrelin, obestatin, cholecystokinin, GLP-1(6-37)-NH₂, NPY and α-MSH.

In various exemplary embodiments, the biomarker panel comprises or consists of total ghrelin, obestatin, cholecystokinin, GLP-1(6-37)-NH₂, NPY, α-MSH and 1, 2, 3, 4 or more additional biomarkers. In various exemplary embodiments, the biomarker panel comprises or consists of any combination of total ghrelin, obestatin, cholecystokinin, GLP-1(6-37)-NH₂, NPY, α-MSH and 1, 2, 3, 4 or more additional biomarkers.

In various exemplary embodiments, the biomarker panel comprises ghrelin (e.g. total ghrelin), obestatin, cholecystokinin, GLP-1 (e.g. GLP-1(6-37)-NH₂), NPY and proopiomelanocortin (e.g., α-MSH). As discussed below, in general, panels comprising both protein and nucleic acid will be detected on two different substrates. In various additional exemplary embodiments, the biomarker panel comprises any combination of ghrelin (e.g. total ghrelin), obestatin, cholecystokinin, GLP-1 (e.g. GLP-1(6-37)-NH₂), NPY and proopiomelanocortin (e.g., α-MSH). In various exemplary embodiments, the biomarker panel consists of ghrelin (e.g. total ghrelin), obestatin, cholecystokinin, GLP-1 (e.g. GLP-1(6-37)-NH₂), NPY and proopiomelanocortin (e.g., α-MSH). In various exemplary embodiments, the biomarker panel consists of any combination of ghrelin (e.g. total ghrelin), obestatin, cholecystokinin, GLP-1 (e.g. GLP-1(6-37)-NH₂), NPY and proopiomelanocortin (e.g., α-MSH).

In various exemplary embodiments, the biomarker panel comprises or consists of ghrelin (e.g. total ghrelin), obestatin, cholecystokinin, GLP-1 (e.g. GLP-1(6-37)-NH₂), NPY, proopiomelanocortin (e.g., α-MSH) and 1, 2, 3, 4 or more additional biomarkers. In various exemplary embodiments, the biomarker panel comprises or consists of any combination of ghrelin (e.g. total ghrelin), obestatin, cholecystokinin, GLP-1 (e.g. GLP-1(6-37)-NH₂), NPY, proopiomelanocortin (e.g., α-MSH) and 1, 2, 3, 4 or more additional biomarkers.

### Measurement and Detection of Biomarkers

Biomarkers generally can be measured and detected through a variety of assays, methods and detection systems known to one of skill in the art. The term “measuring,” “detecting,” or “taking a measurement” refers to a quantitative or qualitative determination of a property or characteristic of an entity, e.g., quantifying the amount or the activity level of a molecule. The term “concentration” or “level” can refer to an absolute or relative quantity. Measuring a molecule may also include determining the absence or presence of the molecule. A measurement may refer to one observation under a set of conditions or an equally- or differently-weighted average of a plurality of observations under the same set of conditions. Thus, in various embodiments, a measurement of the concentration of a biomarker is derived from one observation of the concentration, and in various embodiments, a measurement of a biomarker is derived from an equally- or differently-weighted average of a plurality of observations of the concentration. In various embodiments, measuring a biomarker panel comprises measuring the concentrations of each member of the biomarker panel in a sample.

Various methods include but are not limited to refractive index spectroscopy (RI), ultra-violet spectroscopy (UV), fluorescence analysis, radiochemical analysis, near-infrared spectroscopy (near-IR), infrared (IR) spectroscopy, nuclear magnetic resonance spectroscopy (NMR), light scattering analysis (LS), mass spectrometry, pyrolysis mass spectrometry, nephelometry, dispersive Raman spectroscopy, gas chromatography, liquid chromatography, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, colorimetry and surface plasmon resonance (such as according to systems provided by Biacore Life Sciences). See also WO/2004/056456 and WO/2004/088309. In this regard, biomarkers can be measured using the above-mentioned detection methods, or other methods known to the skilled artisan. Other biomarkers can be similarly detected using reagents that are specifically designed or tailored to detect them.

Different types of biomarkers and their measurements can be combined in the compositions and methods of the present invention. In various embodiments, the protein form of the biomarkers is measured. In various embodiments, the nucleic acid form of the biomarkers is measured. In exemplary embodiments, the nucleic acid form is mRNA. In various embodiments, measurements of protein biomarkers are used in conjunction with measurements of nucleic acid biomarkers.

Using sequence information provided by the database entries for the biomarker sequences, expression of the biomarker sequences can be detected (if present) and measured using known techniques. For example, sequences in sequence database entries or sequences disclosed herein can be used to construct probes for detecting biomarker RNA sequences in, e.g., Northern blot hybridization analyses or methods which specifically, and, preferably, quantitatively amplify specific nucleic acid sequences. As another example, the sequences can be used to construct primers for specifically amplifying the biomarker sequences in, e.g., amplification-based detection methods such as reverse-transcription based polymerase chain reaction (RT-PCR). When alterations in gene expression are associated with gene amplification, deletion, polymorphisms and mutations, sequence comparisons in test and reference populations can be made by comparing relative amounts of the examined DNA sequences in the test and reference cell populations. In addition to Northern blot and RT-PCR, RNA can also be measured using, for example, other target amplification methods (e.g., transcription-mediated amplification (TMA), strand displacement amplification (SDA), nucleic acid sequence based amplification (NASBA) and real time PCR), signal amplification methods (e.g., bDNA), nuclease protection assays, in situ hybridization and the like.

Thus, in one aspect, the invention provides a probe set comprising or consisting of a plurality of probes for detecting a biomarker panel. In one embodiment, a probe set comprises or consists of a capture binding ligand selective for ghrelin (e.g. total ghrelin), a capture binding ligand selective for obestatin, a capture binding ligand selective for cholecystokinin, a capture binding ligand selective for GLP-1 (e.g. GLP-1(6-37)-NH₂), a capture binding ligand selective for NPY and a capture binding ligand selective for proopiomelanocortin (e.g., α-MSH).

In one aspect, the invention provides a primer set comprising or consisting of one or more primers (e.g., one or more primer pairs) for amplifying a nucleic acid form of a biomarker for detection.

As further defined below, a ligand that “specifically binds” or “selectively binds” or is “selective for” a biomarker means that the ligand binds the biomarker with specificity sufficient to differentiate between the biomarker and other components or contaminants of the sample.

Of particular interest for the measurement of biomarkers in the present invention are biochip assays. By “biochip” or “chip” herein is meant a composition generally comprising a solid support or substrate to which a capture ligand (also called an adsorbent, affinity reagent or binding ligand, or when nucleic acid is measured, a capture probe) is attached and can bind either proteins, nucleic acids or both. Generally, where a biochip is used for measurements of protein and nucleic acid biomarkers, the protein biomarkers are measured on a chip separate from that used to measure the nucleic acid biomarkers. For nonlimiting examples of additional platforms and methods useful for measuring nucleic acids, see US/2006/0275782, US/2005/0064469 and DE10201463. In various embodiments, biomarkers are measured on the same platform, such as on one chip. In various embodiments, biomarkers are measured using different platforms and/or different experimental runs.

In one aspect, the invention provides a composition comprising a solid support comprising one or more capture ligands, each selective for a different biomarker of a biomarker panel. In various embodiments, a capture ligand is referred to as a capture binding ligand, which can be, for example, an antibody. In various embodiments, a capture ligand is referred to as a capture probe, which can be, for example, a nucleic acid. In various embodiments, the composition further comprises a soluble binding ligand for one or more biomarkers of a biomarker panel. In one aspect, the invention provides methods of assaying a sample comprising contacting the sample with a solid support comprising one or more capture ligands, each selective for a different biomarker of a biomarker panel, and measuring each of the biomarkers of the biomarker panel.

By “binding ligand,” “capture binding ligand,” “capture binding species,” “capture probe” or “capture ligand” herein is meant a compound that is used to detect the presence of or to quantify, relatively or absolutely, a target analyte, target species or target sequence (all used interchangeably) and that will bind to the target analyte, target species or target sequence. Generally, the capture binding ligand or capture probe allows the attachment of a target species or target sequence to a solid support for the purposes of detection as further described herein. Attachment of the target species to the capture binding ligand may be direct or indirect. In exemplary embodiments, the target species is a biomarker. As will be appreciated by those in the art, the composition of the binding ligand will depend on the composition of the biomarker. Binding ligands for a wide variety of biomarkers are known or can be readily found using known techniques. For example, when the biomarker is a protein, the binding ligands include proteins (particularly including antibodies or fragments thereof (FAbs, etc.) as discussed further below) or small molecules. The binding ligand may also have cross-reactivity with proteins of other species. Antigen-antibody pairs, receptor-ligands, and carbohydrates and their binding partners are also suitable analyte-binding ligand pairs. In various embodiments, the binding ligand may be nucleic acid. Nucleic acid binding ligands find particular use when proteins are the targets; alternatively, as is generally described in U.S. Pat. Nos. 5,270,163; 5,475,096; 5,567,588; 5,595,877; 5,637,459; 5,683,867; 5,705,337 and related patents, hereby incorporated by reference, nucleic acid “aptamers” can be developed for binding to virtually any biomarker. Nucleic acid binding ligands also find particular use when nucleic acids are binding targets. There is a wide body of literature relating to the development of binding partners based on combinatorial chemistry methods. In these embodiments, when the binding ligand is a nucleic acid, preferred compositions and techniques are outlined in WO/1998/020162, hereby incorporated by reference.

Capture binding ligands that are useful in the present invention may be “selective” for, “specifically bind” or “selectively bind” their target, such as a protein. Typically, specific or selective binding can be distinguished from non-specific or non-selective binding when the dissociation constant (K_{D}) is less than about 1×10⁻⁵ M, less than about 1×10⁻⁶ M or less than about 1×10⁻⁷ M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

A capture binding ligand that is selective for “total” forms of a biomarker may be selective for each individual form that is part of the total. For example, a capture binding ligand selective for total ghrelin can be selective for the acyl and desacyl forms. Kits comprising capture binding ligands for measuring total ghrelin are known in the art.

In various exemplary embodiments, the capture binding ligand is an antibody. These embodiments are particularly useful for the detection of the protein form of a biomarker.

Detecting or measuring the concentration (e.g. to determine transcription level) of a biomarker involves binding of the biomarker to a capture binding ligand, generally referred to herein as a “capture probe” when the nucleic acid form (e.g. mRNA) of the biomarker is to be detected on a solid support. In that sense, the biomarker is a target sequence. The term “target sequence” or “target nucleic acid” herein means a nucleic acid sequence that may be a portion of a gene, a regulatory sequence, genomic DNA, cDNA, RNA including mRNA and rRNA, or others. As is outlined herein, the target sequence may be a target sequence found directly in a sample. The target sequence may in some embodiments be a secondary target such as a product of an amplification reaction such as PCR etc. In some embodiments, measuring a nucleic acid can thus refer to measuring the complement of the nucleic acid. It may be any length, with the understanding that longer sequences are more specific.

Capture probes that “selectively bind” (i.e., are “complementary” or “substantially complementary”) to or are “selective for” a target nucleic acid find use in the present invention. “Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single stranded RNA or DNA molecules may be said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least about 14 to about 25 nucleotides, preferably at least about 75%, more preferably at least about 90% complementary. See, generally, M. Kanehisa, Nucleic Acids Res., 2004, 12: 203.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. In one embodiment, stable duplex means that a duplex structure is not destroyed by a stringent wash, e.g. conditions including temperature of about 5° C. less that the Tₘ of a strand of the duplex and low monovalent salt concentration, e.g. less than 0.2 M, or less than 0.1 M. “Perfectly matched” in reference to a duplex means that the poly- or oligonucleotide strands making up the duplex form a double stranded structure with one another such that every nucleotide in each strand undergoes Watson-Crick basepairing with a nucleotide in the other strand. The term “duplex” includes the pairing of nucleoside analogs, such as deoxyinosine, nucleosides with 2-aminopurine bases, PNAs, and the like, that may be employed. A “mismatch” in a duplex between two oligonucleotides or polynucleotides means that a pair of nucleotides in the duplex fails to undergo Watson-Crick bonding.

The target sequence may also comprise different target domains; for example, a first target domain of the sample target sequence may hybridize to a first capture probe, a second target domain may hybridize to a label probe (e.g. a “sandwich assay” format), etc. The target domains may be adjacent or separated as indicated. Unless specified, the terms “first” and “second” are not meant to confer an orientation of the sequences with respect to the 5′-3′ orientation of the target sequence. For example, assuming a 5′-3′ orientation of the target sequence, the first target domain may be located either 5′ to the second domain, or 3′ to the second domain.

When nucleic acids are used as the target analyte, the assays of the invention can take on a number of embodiments. In one embodiment, the assays are done in a solution format. In one embodiment, end-point or real time PCR formats are used, as are well known in the art. These assays can be done either as a panel, in individual tubes or wells, or as multiplex assays, using sets of primers and different labels within a single tube or well. qPCR techniques relying on 5′ nuclease assays using FRET probes or intercalating dyes such as SYBR Green can also be used for nucleic acid targets. In addition to PCR-based solution formats, other formats can be utilized, including, but not limited to for example ligation based assays utilizing FRET dye pairs. In this embodiment, only upon ligation of two (or more) probes hybridized to the target sequence is a signal generated.

In many embodiments, the assays are done on a solid support, utilizing a capture probe associated with the surface. As discussed herein, the capture probes (or capture binding ligands, as they are sometimes referred to) can be covalently attached to the surface, for example using capture probes terminally modified with functional groups, for example amino groups, that are attached to modified surfaces such as silanized glass. Alternatively, non-covalent attachment, such as electrostatic, hydrophobic/hydrophilic adhesion can be utilized. As is appreciated by those in the art and discussed herein, a large number of attachments are possible on a wide variety of surfaces.

In one embodiment, the target sequence comprises a detectable label, as described herein. In this embodiment, the label is generally added to the target sequence during amplification of the target in one of two ways: either labeled primers are utilized during the amplification step or labeled dNTPs are used, both of which are well known in the art.

The detectable label can either be a primary or secondary label as discussed herein. For example, in one embodiment, the label on the primer and/or a dNTP is a primary label such as a fluorophore. In other words, a primary label produces a detectable signal that can be directly detected. By “label” or “labeled” herein is meant that a compound has at least one molecule, element, isotope or chemical compound attached to enable the detection of the compound. In general, labels fall into four classes: a) isotopic labels, which may be radioactive or heavy isotopes; b) magnetic, electrical, thermal; c) colored or luminescent dyes; and d) enzymes; although labels include particles such as magnetic particles as well. The dyes may be chromophores or phosphors but are preferably fluorescent dyes, which due to their strong signals provide a good signal-to-noise ratio for decoding. Suitable dyes for use in the invention include, but are not limited to, fluorescent lanthanide complexes, including those of europium and terbium, fluorescein, rhodamine, tetramethylrhodamine, eosin, erythrosin, coumarin, methyl-coumarins, pyrene, Malacite green, stilbene, Lucifer Yellow, Cascade Blue, Texas Red, Alexa dyes and others described in Molecular Probes Handbook (6th ed.) by Richard P. Haugland. Additional labels include nanocrystals or Q-dots as described in U.S. Pat. No. 6,544,732.

Alternatively, the label may be a secondary label, such as biotin or an enzyme. A secondary label requires additional reagents that lead to the production of a detectable signal. A secondary label is one that is indirectly detected; for example, a secondary label can bind or react with a primary label for detection, can act on an additional product to generate a primary label (e.g. enzymes), or may allow the separation of the compound comprising the secondary label from unlabeled materials, etc. Secondary labels include, but are not limited to, one of a binding partner pair; chemically modifiable moieties; nuclease inhibitors, enzymes such as horseradish peroxidase, alkaline phosphatases, lucifierases, etc. Secondary labels can also include additional labels.

In one embodiment, the primers or dNTPs are labeled with biotin, and then a streptavidin/label complex is added. In one embodiment, the streptavidin/label complex contains a label such as a fluorophore. In an alternative embodiment, the streptavidin/label complex comprises an enzymatic label. For example, the label complex can comprise horseradish peroxidase, and upon addition of a precipitating agent, such as TMB, the action of the horseradish peroxidase causes an optically detectable precipitation reaction. This has a particular benefit in that the optics for detection does not require the use of a fluorimeter or other detector, which can add to the expense of carrying out the methods.

In various embodiments, the secondary label is a binding partner pair. For example, the label may be a hapten or antigen, which will bind its binding partner. Suitable binding partner pairs include, but are not limited to: antigens (such as a polypeptide) and antibodies (including fragments thereof (FAbs, etc.)); other polypeptides and small molecules, including biotin/streptavidin; enzymes and substrates or inhibitors; other protein-protein interacting pairs; receptor-ligands; and carbohydrates and their binding partners. Nucleic acid-nucleic acid binding proteins pairs are also useful. In general, the smaller of the pair is attached to the NTP for incorporation into the primer. Preferred binding partner pairs include, but are not limited to, biotin (or imino-biotin) and streptavidin, digeoxinin and Abs, and Prolinx™ reagents.

In the sandwich formats of the invention, an enzyme serves as the secondary label, bound to the soluble capture ligand. Of particular use in some embodiments is the use of horseradish peroxidase, which when combined with a precipitating agent such as 3,3′,5,5′-tetramethylbenzidine (TMB) forms a colored precipitate which is then detected. In some cases, the soluble capture ligand comprises biotin, which is then bound to a enzyme-streptavidin complex and forms a colored precipitate with the addition of TMB.

Thus, in various embodiments, the detectable label or detectable marker is a conjugated enzyme (for example, horseradish peroxidase). In various embodiments, the system relies on detecting the precipitation of a reaction product or on a change in, for example, electronic properties for detection. In various embodiments, none of the compounds comprises a label.

In alternate embodiments, the solid phase assay relies on the use of a labeled soluble capture ligand, sometimes referred to as a “label probe” or “signaling probe” when the target analyte is a nucleic acid. In this format, the assay is a “sandwich” type assay, where the capture probe binds to a first domain of the target sequence and the label probe binds to a second domain. In this embodiment, the label probe can also be either a primary (e.g. a fluorophore) or a secondary (biotin or enzyme) label. In one embodiment, the label probe comprises biotin, and a streptavidin/enzyme complex is used, as discussed herein. As above, for example, the complex can comprise horseradish peroxidase, and upon addition of TMB, the action of the horseradish peroxidase causes an optically detectable precipitation reaction t.

In embodiments finding particular use herein, a sandwich format is utilized, in which target species are unlabeled. In these embodiments, a “capture” or “anchor” binding ligand is attached to the detection surface as described herein, and a soluble binding ligand (frequently referred to herein as a “signaling probe,” “label probe” or “soluble capture ligand”) binds independently to the target species and either directly or indirectly comprises at least one label or detectable marker.

As used herein, the term “fluorescent signal generating moiety” or “fluorophore” refers to a molecule or part of a molecule that absorbs energy at one wavelength and re-emits energy at another wavelength. Fluorescent properties that can be measured include fluorescence intensity, fluorescence lifetime, emission spectrum characteristics, energy transfer, and the like.

Signals from single molecules can be generated and detected by a number of detection systems, including, but not limited to, scanning electron microscopy, near field scanning optical microscopy (NSOM), total internal reflection fluorescence microscopy (TIRFM), and the like. Abundant guidance is found in the literature for applying such techniques for analyzing and detecting nanoscale structures on surfaces, as evidenced by the following references that are incorporated by reference: Reimer et al, editors, Scanning Electron Microscopy: Physics of Image Formation and Microanalysis, 2nd Edition (Springer, 1998); Nie et al, Anal. Chem., 78: 1528-1534 (2006); Hecht et al, Journal Chemical Physics, 112: 7761-7774 (2000); Zhu et al, editors, Near-Field Optics: Principles and Applications (World Scientific Publishing, Singapore, 1999); Drmanac, WO/2004/076683; Lehr et al, Anal. Chem., 75: 2414-2420 (2003); Neuschafer et al, Biosensors & Bioelectronics, 18: 489-497 (2003); Neuschafer et al, U.S. Pat. No. 6,289,144; and the like.

Thus, a detection system for fluorophores includes any device that can be used to measure fluorescent properties as discussed above. In various embodiments, the detection system comprises an excitation source, a fluorophore, a wavelength filter to isolate emission photons from excitation photons and a detector that registers emission photons and produces a recordable output, in some embodiments as an electrical signal or a photographic image. Examples of detection devices include without limitation spectrofluorometers and microplate readers, fluorescence microscopes, fluorescence scanners (including e.g. microarray readers) and flow cytometers.

The term “solid support” or “substrate” refers to any material that can be modified to contain discrete individual sites appropriate for the attachment or association of a capture binding ligand. Suitable substrates include metal surfaces such as gold, electrodes, glass and modified or functionalized glass, plastics (including acrylics, polystyrene and copolymers of styrene and other materials, polypropylene, polyethylene, polybutylene, polycarbonate, polyurethanes, Teflon, derivatives thereof, etc.), polysaccharides, nylon or nitrocellulose, resins, mica, silica or silica-based materials including silicon and modified silicon, carbon, metals, inorganic glasses, fiberglass, ceramics, GETEK (a blend of polypropylene oxide and fiberglass) and a variety of other polymers. Of particular use in the present invention are the ClonDiag™ materials described below.

In one aspect, the invention provides a solid support comprising or consisting of capture binding ligands selective for the protein form of the members of a biomarker panel. In one aspect, the invention provides a solid support comprising or consisting of capture probes selective for the nucleic acid form of the members of a biomarker panel.

Frequently, the surface of a biochip comprises a plurality of addressable locations, each of which comprises a capture binding ligand. An “array location,” “addressable location,” “pad” or “site” herein means a location on the substrate that comprises a covalently attached capture binding ligand. An “array” herein means a plurality of capture binding ligands in a regular, ordered format, such as a matrix. The size of the array will depend on the composition and end use of the array. Arrays containing from about two or more different capture binding ligands to many thousands can be made. Generally, the array will comprise a plurality of types of capture binding ligands depending on the end use of the array. In the present invention, the array can include controls, replicates of the markers and the like. Exemplary ranges are from about 3 to about 50. In some embodiments, the compositions of the invention may not be in array format; that is, for some embodiments, compositions comprising a single capture ligand may be made as well. In addition, in some arrays, multiple substrates may be used, either of different or identical compositions. Thus for example, large arrays may comprise a plurality of smaller substrates.

Accordingly, in one aspect, the invention provides a composition comprising a solid support comprising a capture binding ligand for each biomarker of a biomarker panel. In various embodiments, the capture binding ligand is an antibody. In various embodiments, the composition further comprises a soluble binding ligand for each biomarker of a biomarker panel.

A number of different biochip array platforms as known in the art may be used. For example, the compositions and methods of the present invention can be implemented with array platforms such as GeneChip (Affymetrix), CodeLink Bioarray (Amersham), Expression Array System (Applied Biosystems), SurePrint microarrays (Agilent), Sentrix LD BeadChip or Sentrix Array Matrix (Illumina) and Verigene (Nanosphere).

In various exemplary embodiments, detection and measurement of biomarkers utilizes colorimetric methods and systems in order to provide an indication of binding of a target analyte or target species. In colorimetric methods, the presence of a bound target species such as a biomarker will result in a change in the absorbance or transmission of light by a sample or substrate at one or more wavelengths. Detection of the absorbance or transmission of light at such wavelengths thus provides an indication of the presence of the target species.

A detection system for colorimetric methods includes any device that can be used to measure colorimetric properties as discussed above. Generally, the device is a spectrophotometer, a colorimeter or any device that measures absorbance or transmission of light at one or more wavelengths. In various embodiments, the detection system comprises a light source; a wavelength filter or monochromator; a sample container such as a cuvette or a reaction vial; a detector, such as a photoresistor, that registers transmitted light; and a display or imaging element. In some embodiments, a change in the colorimetric properties of a sample can be detected directly by the naked eye, i.e., by direct visual inspection.

In various exemplary embodiments, a ClonDiag chip platform is used for the colorimetric detection of biomarkers. In various embodiments, a ClonDiag ArrayTube (AT) is used. One unique feature of the ArrayTube is the combination of a micro probe array (the biochip) and micro reaction vial. In various embodiments, where a target sequence is a nucleic acid, detection of the target sequence is done by amplifying and biotinylating the target sequence contained in a sample and optionally digesting the amplification products. The amplification product is then allowed to hybridize with probes contained on the ClonDiag chip. A solution of a streptavidin-enzyme conjugate, such as Poly horseradish peroxidase (HRP) conjugate solution, is contacted with the ClonDiag chip. After washing, a dye solution such as o-dianisidine substrate solution is contacted with the chip. Oxidation of the dye results in precipitation that can be detected colorimetrically. Further description of the ClonDiag platform is found in Monecke S, Slickers P, Hotzel H et al., Clin Microbiol Infect 2006, 12: 718-728; Monecke S, Berger-Bächi B, Coombs C et al., Clin Microbiol Infect 2007, 13: 236-249; Monecke S, Leube I and Ehricht R, Genome Lett 2003, 2: 106-118; German Patent DE 10201463; US Publication US/2005/0064469 and ClonDiag, ArrayTube (AT) Experiment Guideline for DNA-Based Applications, version 1.2, 2007, all incorporated by reference in their entirety. Use of the ClonDiag platform for genotyping is described in Sachse K et al., BMC Microbiology 2008, 8: 63; Monecke S and Ehricht R, Clin Microbiol Infect 2005, 11: 825-833; and Monecke S et al., Clin Microbiol Infect 2008, 14(6): 534-545. One of skill in the art will appreciate that numerous other dyes that react with a peroxidase can be utilized to produce a colorimetric change, such as 3,3′,5,5′-tetramethylbenzidine (TMB). For information on specific assay protocols, see www.clondiag.com/technologies/publications.php. Such dyes may be referred to as a precipitating agent herein.

In various embodiments, where a target species is a protein, the ArrayTube biochip comprises capture binding ligands such as antibodies. A sample is contacted with the biochip, and any target species present in the sample is allowed to bind to the capture binding ligand antibodies. A soluble capture binding ligand or a detection compound such as a horseradish peroxidase conjugated antibody is allowed to bind to the target species. A dye, such as TMB, is then added and allowed to react with the horseradish peroxidase, causing precipitation and a color change that is detected by a suitable detection device. Further description of protein detection using ArrayTube is found in, for example, Huelseweh B, Ehricht R and Marschall H-J, Proteomics, 2006, 6, 2972-2981; and ClonDiag, ArrayTube (AT) Experiment Guideline for Protein-Based Applications, version 1.2, 2007, all incorporated by reference in their entirety.

Transmission detection and analysis is performed with a ClonDiag AT reader instrument. Suitable reader instruments and detection devices include the ArrayTube Workstation ATS and the ATR 03.

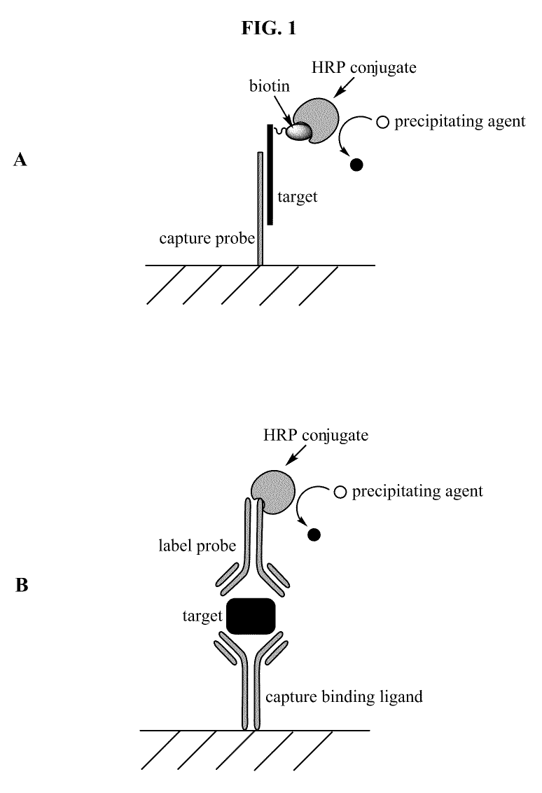

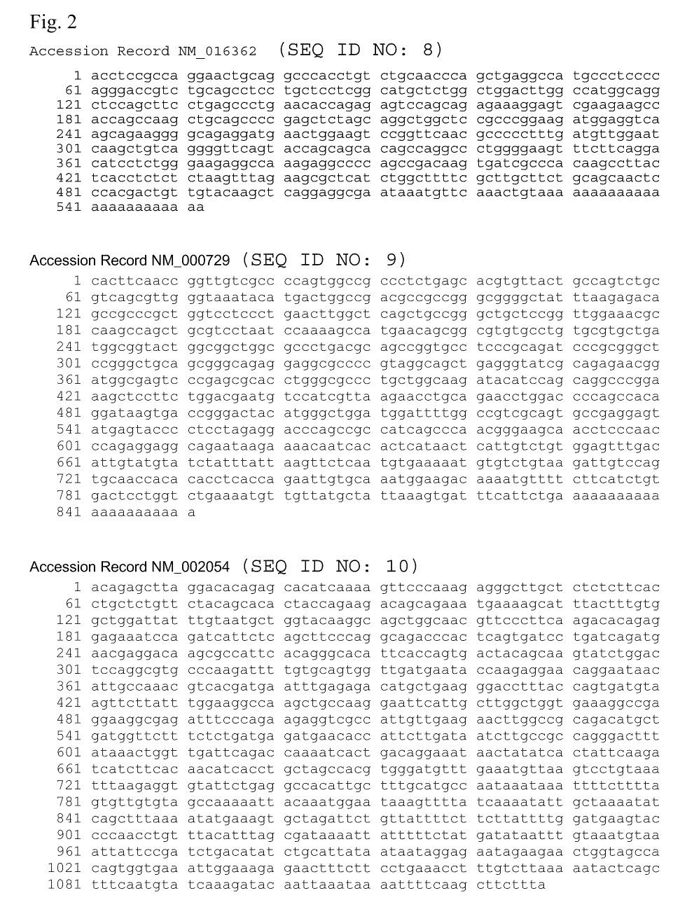

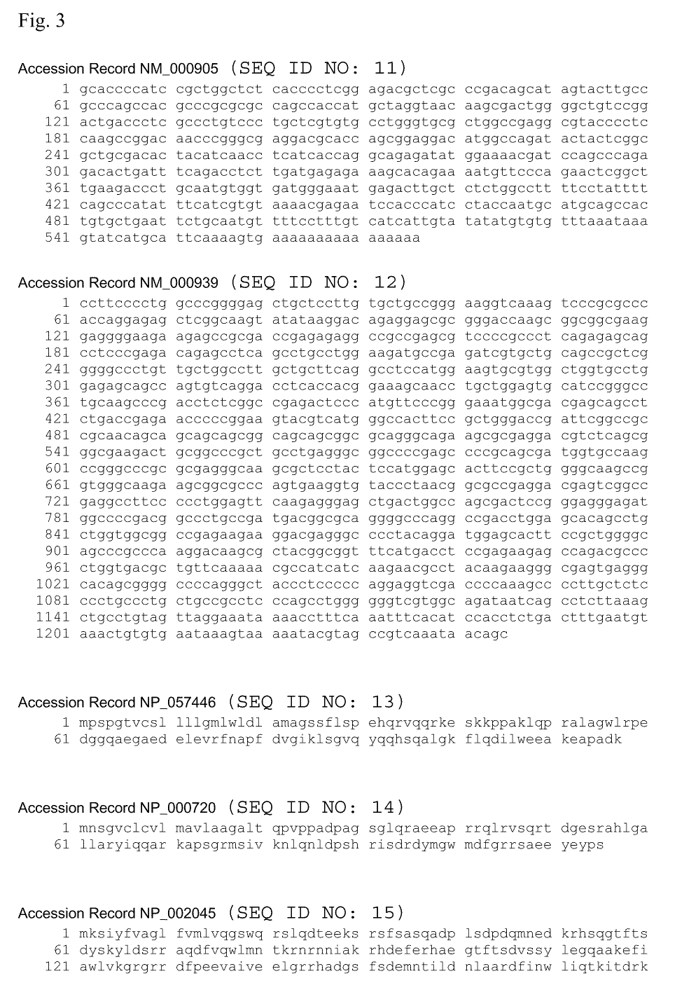

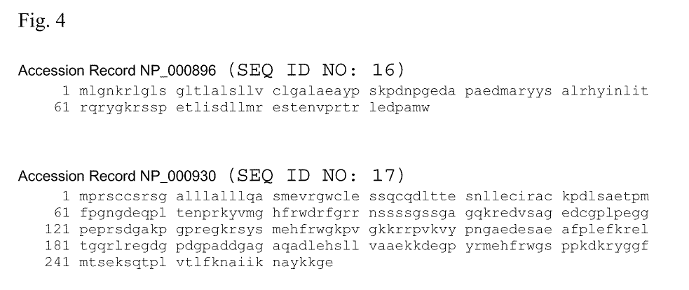

A schematic of example assay configurations that can used for detection is shown in FIGS. 1A and 1B. FIG. 1A shows a configuration that can be used to detect a nucleic acid target. A capture probe is attached to a solid support, and a target labeled with biotin binds to the capture probe. A horseradish peroxidase (HRP) conjugate binds to the biotin, and when a soluble precipitating agent contacts the HRP, a visible precipitate is created. FIG. 1B shows a configuration that can be used to detect a polypeptide target, following a similar principle. In FIG. 1B, the capture binding ligand and label probes are depicted as antibodies. The HRP conjugate can be directly bound to the label probe or via a biotin-streptavidin linkage. These configurations are particularly suited for use with the ClonDiag platform.

In addition to ArrayTube, the ClonDiag ArrayStrip (AS) can be used. The ArrayStrip provides a 96-well format for high volume testing. Each ArrayStrip consists of a standard 8-well strip with a microarray integrated into the bottom of each well. Up to 12 ArrayStrips can be inserted into one microplate frame enabling the parallel multiparameter testing of up to 96 samples. The ArrayStrip can be processed using the ArrayStrip Processor ASP, which performs all liquid handling, incubation, and detection steps required in array based analysis. In various embodiments, where a protein is detected, a method of using the ArrayStrip to detect the protein comprises conditioning the AS array with buffer or blocking solution; loading of up to 96 sample solutions in the AS wells to allow for binding of the protein; 3× washing; conjugating with a secondary antibody linked to HRP; 3× washing; precipitation staining with TMB; and AS array imaging and optional data storage.

Those skilled in the art will be familiar with numerous additional immunoassay formats and variations thereof which may be useful for carrying out the method disclosed herein. See generally E. Maggio, Enzyme-Immunoassay, (CRC Press, Inc., Boca Raton, Fla., 1980); see also U.S. Pat. Nos. 4,727,022; 4,659,678; 4,376,110; 4,275,149; 4,233,402; and 4,230,767.

In general, immunoassays carried out in accordance with the present invention may be homogeneous assays or heterogeneous assays. In a homogeneous assay the immunological reaction usually involves the specific antibody (e.g., anti-biomarker protein antibody), a labeled analyte, and the sample of interest. The signal arising from the label is modified, directly or indirectly, upon the binding of the antibody to the labeled analyte. Both the immunological reaction and detection of the extent thereof can be carried out in a homogeneous solution. Immunochemical labels which may be employed include free radicals, radioisotopes, fluorescent dyes, enzymes, bacteriophages, or coenzymes.

In a heterogeneous assay approach, the reagents are usually the sample, the antibody, and means for producing a detectable signal. Samples as described above may be used. The antibody can be immobilized on a support, such as a bead (such as protein A and protein G agarose beads), plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the sample. Means for producing a detectable signal include the use of radioactive labels, fluorescent labels, or enzyme labels. For example, if the antigen to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays include immunoblotting, immunofluorescence methods, immunoprecipitation, chemiluminescence methods, electrochemiluminescence (ECL) or enzyme-linked immunoassays.

Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein, rhodamine) in accordance with known techniques.

As used herein, the term “antibody” means a protein comprising one or more polypeptides substantially encoded by all or part of the recognized immunoglobulin genes. The recognized immunoglobulin genes, for example in humans, include the kappa (κ), lambda (λ) and heavy chain genetic loci, which together compose the myriad variable region genes, and the constant region genes mu (μ), delta (δ), gamma (γ), epsilon (δ) and alpha (α), which encode the IgM, IgD, IgG, IgE, and IgA isotypes respectively. Antibody herein is meant to include full length antibodies and antibody fragments, and may refer to a natural antibody from any organism, an engineered antibody or an antibody generated recombinantly for experimental, therapeutic or other purposes as further defined below. Antibody fragments include Fab, Fab′, F(ab′)₂, Fv, scFv or other antigen-binding subsequences of antibodies and can include those produced by the modification of whole antibodies or those synthesized de novo using recombinant DNA technologies. The term “antibody” refers to both monoclonal and polyclonal antibodies. Antibodies can be antagonists, agonists, neutralizing, inhibitory or stimulatory.

The invention further provides kits for performing any of the methods disclosed herein for a number of medical (including diagnostic and therapeutic), industrial, forensic and research applications. In some embodiments, the kits are for determining therapy response in a subject. Kits may comprise a portable carrier, such as a box, carton, tube or the like, having in close confinement therein one or more containers, such as vials, tubes, ampoules, bottles, pouches, envelopes and the like. In various embodiments, a kit comprises one or more components selected from one or more media or media ingredients and reagents for the measurement of the various biomarkers and biomarker panels disclosed herein. For example, kits of the invention may also comprise, in the same or different containers, in any combination, one or more DNA polymerases, one or more primers, one or more probes, one or more binding ligands, one or more suitable buffers, one or more nucleotides (such as deoxynucleoside triphosphates (dNTPs) and preferably labeled dNTPs), one or more detectable labels and markers and one or more solid supports, any of which is described herein. The components may be contained within the same container, or may be in separate containers to be admixed prior to use. The kits of the present invention may also comprise one or more instructions or protocols for carrying out the methods of the present invention. The kits may comprise a detector for detecting a signal generated through use of the components of the invention in conjunction with a sample. The kits may also comprise a computer or a component of a computer, such as a computer-readable storage medium or device. Examples of storage media include, without limitation, optical disks such as CD, DVD and Blu-ray Discs (BD); magneto-optical disks; magnetic media such as magnetic tape and internal hard disks and removable disks; semi-conductor memory devices such as EPROM, EEPROM and flash memory; and RAM. The computer-readable storage medium may comprise software encoding references to the various therapies and treatment regimens disclosed herein. The software may be interpreted by a computer to provide the practitioner with treatments according to various measured concentrations of biomarkers as provided herein. In various embodiments, the kit comprises a biomarker assay involving a lateral-flow-based point-of-care rapid test with detection of risk thresholds, or a biochip with quantitative assays for the constituent biomarkers. Generally, any of the methods disclosed herein can comprise using any of the kits (comprising primers, probes, labels, ligands, reagents and solid supports in any combination) disclosed herein.

In one aspect, the invention provides a kit comprising a solid support comprising or consisting of capture binding ligands selective for the protein form of the members of a biomarker panel. In one aspect, the invention provides a kit comprising a solid support comprising or consisting of capture probes selective for the nucleic acid form of the members of a biomarker panel. In one aspect, the invention provides a kit comprising (a) a solid support comprising or consisting of capture binding ligands selective for the protein form of the members of a biomarker panel and (b) a solid support comprising or consisting of capture probes selective for the nucleic acid form of the members of a biomarker panel.

In one aspect, the invention provides use of a kit comprising a solid support comprising probes selective for members of a biomarker panel for determining a second therapy for a subject that has undergone a first therapy, wherein the subject is suffering from a disease. In one embodiment, the use comprises (a) contacting a first sample from the subject with a solid support of the kit; (b) taking a first measurement of the concentrations of the biomarker panel in the first sample; (c) effecting a first therapy on the subject; (d) contacting a second sample from the subject with the solid support of the kit; (e) taking a second measurement of the concentrations of the biomarker panel in the second sample and (f) making a comparison of the first and second measurements.

In one aspect, the invention provides use of a kit comprising a solid support comprising probes selective for members of a biomarker panel for determining whether a subject belongs to a population that would benefit from a second therapy, wherein the subject has undergone a first therapy. In one embodiment, the use comprises (a) contacting a first sample from the subject with a solid support of the kit; (b) taking a first measurement of the concentrations of the biomarker panel in the first sample; (c) effecting a first therapy on the subject; (d) contacting a second sample from the subject with the solid support of the kit; (e) taking a second measurement of the concentrations of the biomarker panel in the second sample and (f) making a comparison of the first and second measurements.

Using any of the methods and compositions described herein, a sample can be assayed to determine concentrations of a biomarker panel. Thus, in one aspect, the invention provides a method of assaying a sample comprising taking a measurement of a biomarker panel in the sample. In one aspect, the invention provides a method of acquiring data relating to a sample comprising taking a measurement of a biomarker panel in the sample. In one aspect, the invention provides a method of measuring analyte concentrations in a sample comprising taking a measurement of a biomarker panel in the sample. Any method or use herein could comprise contacting a sample with a composition comprising a solid support comprising a capture binding ligand or capture probe for each biomarker of a biomarker panel and taking a measurement of the biomarker panel (e.g. to determine biomarker concentrations). Any biomarker panel disclosed herein can be used in these and other methods and uses.

### Methods of Diagnosing and Treating

The compositions and methods of the present invention can be used in the prognosis, diagnosis and treatment of disease in a subject.

A “subject” in the context of the present invention is an animal, preferably a mammal. The mammal can be a human, non-human primate, mouse, rat, dog, cat, horse, or cow, but are not limited to these examples. In various exemplary embodiments, a subject is human and may be referred to as a “patient”. Mammals other than humans can be advantageously used as subjects that represent animal models of a disease or for veterinarian applications. A subject can be one who has been previously diagnosed or identified as having a disease, and optionally has already undergone, or is undergoing, a therapeutic intervention for a disease. Alternatively, a subject can also be one who has not been previously diagnosed as having a disease. For example, a subject can be one who exhibits one or more risk factors for a disease, or one who does not exhibit a disease risk factor, or one who is asymptomatic for a disease. A subject can also be one who is suffering from or at risk of developing a disease. In certain embodiments, the subject can be already undergoing therapy or can be a candidate for therapy.

The invention provides compositions and methods for laboratory and point-of-care tests for measuring biomarkers in a sample from a subject. The invention can be generally applied for a number of different diseases. In exemplary embodiments, the disease is insulin resistance. In exemplary embodiments, the disease is cardiovascular disease or risk. In exemplary embodiments, the disease is atherosclerosis. In exemplary embodiments, the disease is diabetes mellitus. In exemplary embodiments, the disease is obesity.

In exemplary embodiments, the disease is cardiodiabetes. Thus, the panel of biomarkers disclosed herein may find particular use for in diagnosing and treating disorders associated with cardiodiabetes. “Cardiodiabetes” refers to patients with insulin resistance and β-cell dysfunction without elevation of blood glucose who are not identified as suffering from diabetes mellitus. These normoglycemic patients, however, experience the same elevated cardiovascular risk, which is predominantly linked to vascular insulin resistance. A cardiodiabetic subject might not exhibit one or more of the normal symptoms of type 2 diabetes including, but not limited to, hyperglycemia, fatigue, weight gain, excessive eating, poor wound healing and infections. A cardiodiabetic subject is at high risk for cardiovascular disease and may experience events such as myocardial infarction and stroke. That is, diabetes mellitus, cardiodiabetes and metabolic syndrome are phenotypes of a common underlying pathophysiology.

The biomarkers and biomarker panels disclosed herein can be used in methods to diagnose, identify or screen subjects that have, do not have or are at risk for having disease; to monitor subjects that are undergoing therapies for disease; to determine or suggest a new therapy or a change in therapy; to differentially diagnose disease states associated with the disease from other diseases or within sub-classifications of disease; to evaluate the severity or changes in severity of disease in a subject; to stage a subject with the disease and to select or modify therapies or interventions for use in treating a subject with the disease. In an exemplary embodiment, the methods of the present invention are used to identify and/or diagnose subjects who are asymptomatic or presymptomatic for a disease. In this context, “asymptomatic” or “presymptomatic” means not exhibiting the traditional symptoms or enough abnormality for disease. In exemplary embodiments, the subject is normoglycemic.

In one aspect, the invention provides a method of determining a prognosis of a disease in a subject, diagnosing a disease in a subject, or treating a disease in a subject comprises taking a measurement of a biomarker panel in a sample from the subject.

The term “disease status” includes any distinguishable manifestation of the disease, including non-disease. For example, disease status includes, without limitation, the presence or absence of disease, the risk of developing disease, the stage of the disease, the progression of disease (e.g., progress of disease or remission of disease over time), the severity of disease and the effectiveness or response to treatment of disease.

As will be appreciated by those in the art, the biomarkers may be measured in using several techniques designed to achieve more predictable subject and analytical variability. On subject variability, many of the above biomarkers are commonly measured in a fasting state, commonly in the morning, providing a reduced level of subject variability due to both food consumption and metabolism and diurnal variation. All fasting and temporal-based sampling procedures using the biomarkers described herein may be useful for performing the invention. Pre-processing adjustments of biomarker results may also be intended to reduce this effect.

The term “sample” used herein refers to a specimen or culture obtained from a subject and includes fluids, gases and solids including for example tissue. In various exemplary embodiments, the sample comprises blood. A sample could be a fluid obtained from a subject including, for example, whole blood or a blood derivative (e.g. serum, plasma, or blood cells), ovarian cyst fluid, ascites, lymphatic, cerebrospinal or interstitial fluid, saliva, mucous, sputum, sweat, urine, or any other secretion, excretion, or other bodily fluids. As will be appreciated by those in the art, virtually any experimental manipulation or sample preparation steps may have been done on the sample. For example, wash steps may be applied to a sample. In various embodiments, a biomarker panel is measured directly in a subject without the need to obtain a separate sample from the patient.

In one aspect, the invention provides a method of diagnosing a subject for a disease comprising taking a measurement of a biomarker panel in a sample from the subject; and correlating the measurement with the disease. The term “correlating” generally refers to determining a relationship between one type of data with another or with a state. In various embodiments, correlating the measurement with disease comprises comparing the measurement with a reference biomarker profile or some other reference value. In various embodiments, correlating the measurement with disease comprises determining whether the subject is currently in a state of disease.

The quantity or activity measurements of a biomarker panel can be compared to a reference value. Differences in the measurements of biomarkers in the subject sample compared to the reference value are then identified. In exemplary embodiments, the reference value is given by a risk category as described further below.

In various embodiments, the reference value is a baseline value. A baseline value is a composite sample of an effective amount of biomarkers from one or more subjects who do not have a disease, who are asymptomatic for a disease or who have a certain level of a disease. A baseline value can be the concentration of biomarkers measured in a sample obtained from a subject before a therapy is effected on the subject. A baseline value can also comprise the amounts of biomarkers in a sample derived from a subject who has shown an improvement in risk factors of a disease as a result of treatments or therapies. In these embodiments, to make comparisons to the subject-derived sample, the amounts of biomarkers are similarly calculated. A baseline value can also comprise the amounts of biomarkers derived from subjects who have a disease confirmed by an invasive or non-invasive technique, or are at high risk for developing a disease. Optionally, subjects identified as having a disease, or being at increased risk of developing a disease are chosen to receive a therapeutic regimen to slow the progression of a disease, or decrease or prevent the risk of developing a disease. A disease is considered to be progressive (or, alternatively, the treatment does not prevent progression) if the amount of biomarker changes over time relative to the reference value, whereas a disease is not progressive if the amount of biomarkers remains constant over time (relative to the reference population, or “constant” as used herein). The term “constant” as used in the context of the present invention is construed to include changes over time with respect to the reference value.

The biomarkers of the present invention can be used to generate a “reference biomarker profile” of those subjects who do not have a disease according to a certain threshold, are not at risk of having a disease or would not be expected to develop a disease. The biomarkers disclosed herein can also be used to generate a “subject biomarker profile” taken from subjects who have a disease or are at risk for having a disease. The subject biomarker profiles can be compared to a reference biomarker profile to diagnose or identify subjects at risk for developing a disease, to monitor the progression of disease, as well as the rate of progression of disease, and to monitor the effectiveness of disease treatment modalities. The reference and subject biomarker profiles of the present invention can be contained in a machine-readable medium, such as but not limited to, analog tapes like those readable by a VCR; optical media such as CD-ROM, DVD-ROM and the like; and solid state memory, among others.

The biomarker panels of the invention can be used by a practitioner to determine and effect appropriate therapies with respect to a subject given the disease status indicated by measurements of the biomarkers in a sample from the subject. Thus, in one aspect, the invention provides a method of treating a disease in a subject comprising taking a measurement of a biomarker panel in a sample from the subject, and effecting a therapy with respect to the subject. In one embodiment, the concentrations of the biomarkers of the biomarker panel increase or decrease according to the values described herein or stay the same in response to the therapy.

The terms “therapy” and “treatment” may be used interchangeably. In certain embodiments, the therapy can be selected from, without limitation, initiating therapy, continuing therapy, modifying therapy or ending therapy. A therapy also includes any prophylactic measures that may be taken to prevent disease.

In certain embodiments, effecting a therapy comprises administering a disease-modulating drug to a subject. Various examples of suitable disease-modulating drugs are described below. In exemplary embodiments, the disease-modulating drug is an insulin sensitizer. In exemplary embodiments, the disease-modulating drug is a glitazone. In exemplary embodiments, the disease-modulating drug is pioglitazone. In exemplary embodiments, the disease-modulating drug is a GLP-1 analog. Generally, the drug can be a therapeutic or prophylactic used in subjects diagnosed or identified with a disease or at risk of having the disease. In certain embodiments, modifying therapy refers to altering the duration, frequency or intensity of therapy, for example, altering dosage levels. In certain embodiments, a therapy comprises administering a combination of disease-modulating drugs (e.g., combinations including an insulin sensitizer drug) to a subject.

In various embodiments, effecting a therapy comprises causing a subject to make or communicating to a subject the need to make a change in lifestyle, for example, increasing exercise, changing diet, reducing or eliminating smoking and so on. The therapy can also include surgery, for example, bariatric surgery. In various embodiments, effecting a therapy comprises causing a subject to follow or communicating to a subject the need to follow a dietary regimen having a high fiber and low carbohydrate content.

Measurement of biomarker concentrations allows for the course of treatment of a disease to be monitored. The effectiveness of a treatment regimen for a disease can be monitored by detecting one or more biomarkers of a biomarker panel in an effective amount from samples obtained from a subject over time and comparing the amount of biomarkers detected. For example, a first sample can be obtained prior to the subject receiving treatment and one or more subsequent samples are taken after or during treatment of the subject. Changes in biomarker concentrations across the samples may provide an indication as to the effectiveness of the therapy.

To identify therapeutics or drugs that are appropriate for a specific subject, a test sample from the subject can be exposed to a therapeutic agent or a drug, and the concentration of one or more biomarkers can be determined. Biomarker concentrations can be compared to a sample derived from the subject before and after treatment or exposure to a therapeutic agent or a drug, or can be compared to samples derived from one or more subjects who have shown improvements relative to a disease as a result of such treatment or exposure.

### Drug Treatments

In exemplary embodiments, effecting a therapy with respect to a subject comprises administering a disease-modulating drug to the subject. The drug may be in any form suitable for administration to a subject, such forms including salts, prodrugs and solvates. The drug may be formulated in any manner suitable for administration to a subject, often according to various known formulations in the art or as disclosed or referenced herein. For example, the drug may be a component of a pharmaceutical composition comprising the drug and an excipient. Any drug, combination of drugs or formulation thereof disclosed herein may be administered to a subject to treat a disease.

The subject may be treated with one or more disease-modulating drugs until altered concentrations of the measured biomarkers return to a baseline value measured in a population not suffering from the disease, experiencing a less severe stage or form of a disease or showing improvements in disease biomarkers as a result of treatment with a disease-modulating drug. Additionally, improvements related to a changed concentration of a biomarker or clinical parameter may be the result of treatment with a disease-modulating drug and may include, for example, a reduction in body mass index (BMI), a reduction in total cholesterol concentrations, a reduction in LDL concentrations, an increase in HDL concentrations, a reduction in systolic and/or diastolic blood pressure, or combinations thereof.

A number of compounds such as a disease-modulating drug may be used to treat a subject and to monitor progress using the methods of the invention. In certain embodiments, the disease-modulating drug comprises an antiobesity drug, a β-blocker, an angiotensin-converting enzyme (ACE) inhibitor, a diuretic, a calcium channel blocker, an angiotensin II receptor blocker, a antiplatelet agent, an anti-coagulant agent, a sulfonylurea (SU), a biguanide, an insulin, a glitazone (thiazolidinedione (TZD)), a nitrate, a non-steroidal anti-inflammatory agent, a statin, cilostazol, pentoxifylline, buflomedil or naftidrofuryl. In addition, any combination of these drugs may be administered.

The beneficial effects of these and other drugs can be visualized by assessment of clinical and laboratory biomarkers. For example, results from PROactive (Pfützner et al., Expert Review of Cardiovascular Therapy, 2006, 4: 445-459) and recent metanalyses have shown that these surrogate changes may translate into effective reduction of macrovascular risk in patients with type 2 diabetes mellitus.

Insulin sensitizer drugs are particularly useful in the various compositions and methods of the invention. An “insulin sensitizer” as used herein refers to any drug that enhances a subject's response to insulin. Exemplary insulin sensitizers act as agonists to PPAR, in particular to PPARγ. General classes of insulin sensitizers include, without limitation, glitazones (also referred to as thiazolidinediones(TZD)) and glitazars. In some embodiments, metformin is considered to be an insulin sensitizer.

Accordingly, in exemplary embodiments, effecting a therapy comprises administering an insulin sensitizer drug to a subject. Numerous insulin sensitizers are known in the art and are useful in the present invention. Specific examples of insulin sensitizers include pioglitazone, rosiglitazone, netoglitazone (MCC-555), balaglitazone (DRF-2593), rivoglitazone (CS-011), troglitazone, MB-13.1258, 5-(2,4-dioxothiazolidin-5-ylmethyl)-2-methoxy-N[4-(trifluoromethyl)benzyl]benzamide (KRP-297), FK-614, compounds described in WO/1999/058510 (e.g. (E)-4-[4-(5-methyl-2-phenyl-4-oxazolylmethoxy)benzyloxyimino]-4-phenylbutyric acid), aleglitazar, farglitazar (GI-262570), tesaglitazar (AZ-242), ragaglitazar (N,N-622), muraglitazar (BMS-298585), reglitazar (JTT-501), ONO-5816, LM-4156, metaglidasen (MBX-102), naveglitazar (LY-519818), MX-6054, LY-510929, T-131, THR-0921 and the like. See WO/2005/041962 and US/2006/0280794.

In various exemplary embodiments, a glitazone is administered to a subject to treat a disease. In various exemplary embodiments, pioglitazone is administered to a subject. These and other drugs that are administered to treat a subject have been shown to affect concentrations of various biomarkers.

Furthermore, an insulin sensitizer such as pioglitazone may also be administered with other drugs. In various embodiments, pioglitazone is administered with a statin, including but not limited to simvastatin. In various embodiments, pioglitazone may be administered with insulin or a GLP-1 analog, such as exenatide. In various embodiments, pioglitazone may be administered with an oral antidiabetic drug, including but not limited to a sulfonylurea (such as glimepiride), a biguanide (such as metformin), or a DPPIV-inhibitor (such as sitagliptin).

In addition, any of these drugs may be administered alone. Thus, in various embodiments, a glucagon-like peptide 1 (GLP-1) analog is administered to a subject to treat a disease. Examples of GLP-1 analogs include but are not limited to exenatide and liraglutide. GLP-1 analogs have exemplary usefulness in treating various disorders, such as obesity.

In various embodiments, a dipeptidyl peptidase IV (DPPIV) inhibitor is administered to a subject to treat a disease. Examples of DPPIV inhibitors include but are not limited to sitagliptin, vildagliptin and saxagliptin.

In various embodiments, metformin is administered to a subject to treat a disease.

In various embodiments, a glinide is administered to a subject to treat a disease. Examples of glinides include but are not limited to repgalinide and nateglinide.

In various embodiments, a sulfonylurea is administered to a subject to treat a disease. Examples of sulfonylureas include but are not limited to gliclazide and glimepiride.

In various embodiments, an α-glucosidase inhibitor is administered to a subject to treat a disease. An example of an α-glucosidase inhibitor is acarbose.

In various embodiments, an insulin is administered to a subject to treat a disease. The term “insulin” by itself refers to any naturally occurring form of insulin as well as any derivatives and analogs thereof. Different types of insulin may vary in the onset, peak occurrence and duration of their effects. Examples of insulin that may be useful in the present invention include but are not limited to regular human insulin, intermediate acting regular human insulin (e.g., NPH human insulin), Zn-retarded insulin, short acting insulin analog and long acting insulin analog. Examples of Zn-retarded insulin include but are not limited to lente and ultralente. Examples of short-acting insulin analog include but are not limited to lispro, aspart and glulisine. Examples of long-acting insulin analog include but are not limited to glargine and levemir.

In various embodiments, a drug such as an antiobesity drug is administered to a subject. Numerous antiobesity drugs are known and may find use in the present invention. The mechanism of an antiobesity drug can include, without limitation, suppressing appetite, increasing a body's metabolism and interfering with a body's ability to absorb food or components of food (for example, fat). Certain antiobesity drugs such as the pancreatic lipase inhibitors act on the gastrointestinal system, and certain drugs act on the central nervous system. In various embodiments, a subject is administered an antiobesity drug selected from the group consisting of orlistat, sibutramine, metformin, byetta, symlin and rimonabant. In various embodiments, a subject is administered a combination of antiobesity drugs or an antiobesity drug in combination with another drug described herein. In various embodiments, one or more antiobesity drug is combined with one or more treatment regimens such as diet, exercise and so on.

Any drug or combination of drugs disclosed herein may be administered to a subject to treat a disease. The drugs herein can be formulated in any number of ways, often according to various known formulations in the art or as disclosed or referenced herein.

In various embodiments, one or more drug is combined with one or more treatment regimens such as diet, exercise and so on.

### Methods of Determining Treatment Efficacy

Additionally, therapeutic or prophylactic agents (i.e., drugs) suitable for administration to a particular subject can be identified by detecting one or more biomarkers in an effective amount from a sample obtained from a subject and exposing the subject-derived sample to a test compound that determines the amount of the one or more biomarker in the subject-derived sample. Accordingly, treatments or therapeutic regimens for use in subjects having a disease or subjects at risk for developing a disease can be selected based on the amounts of biomarkers in samples obtained from the subjects and compared to a reference value. Two or more treatments or therapeutic regimens can be evaluated in parallel to determine which treatment or therapeutic regimen would be the most efficacious for use in a subject to delay onset, or slow progression of a disease. In various embodiments, a recommendation is made on whether to initiate or continue treatment of a disease. Thus, the biomarker panels of the present invention can be used to determine the efficacy of treatment in a patient or subject.

Accordingly, in one aspect, the invention provides a method of assessing the efficacy of a first therapy on a subject comprising: taking a first measurement of a biomarker panel in a first sample from the subject; effecting the first therapy on the subject; taking a second measurement of the biomarker panel in a second sample from the subject; and making a comparison of the first measurement and the second measurement. In some embodiments, the method further comprises effecting a second therapy on the subject based on the comparison. In exemplary embodiments, the first therapy comprises administering an insulin sensitizer drug to a subject.

In some embodiments, a therapy comprises administering a disease-modulating drug to the subject. In these embodiments, changes in the levels of biomarkers between the first and second measurement allows a physician to either: a) keep the patient on a disease-modulating drug, as the changes in levels of certain biomarkers indicates the drug is working; b) keep the patient on the drug and adjust the dose; c) take the patient off the drug as efficacy is not present; and/or d) add an additional drug to the treatment, whether the patient is kept on the drug or not. Thus, effecting a second therapy in some embodiments comprises making a decision regarding the continued administration of the first disease-modulating drug.

In exemplary embodiments, the first therapy comprises administering a disease-modulating drug according to a first dosage regimen. In some embodiments, the first therapy comprises administering a combination of drugs according to a first dosage regimen. In exemplary embodiments, the combination comprises an insulin sensitizer drug. Thus, the methods of the invention can be used to test the efficacy of a combination of drugs, which can be modified for subsequent therapies according to differences in biomarker panel measurements.

A measurement of a biomarker panel will generally comprise the detection or observation of some characteristic (e.g., concentration (also referred to as a level)) of each member of the biomarker panel. A comparison of a first measurement and a second measurement will indicate a change, if any, in the measured characteristic for the biomarker of interest. A change as used herein may refer to any statistically relevant difference in the characteristic of a biomarker between a first measurement and a second measurement. A statistically relevant difference may be defined by the practitioner or by any art recognized method, and is generally defined herein. For example, a statistically relevant difference may be defined as a difference that surpasses a threshold defined by the practitioner. Thus, in various embodiments, making a comparison of the first measurement and the second measurement comprises determining the difference between the concentration of a biomarker in a first sample determined by the first measurement and the concentration of the biomarker in a second sample determined by the second measurement.

A change may refer to a single quantity, e.g., a 100% difference relative to a first measurement or may refer to a range, e.g., about 50% to about 100% difference or a ≧50% difference relative to a first measurement

A change may occur in either direction relative to a first measurement, i.e., the second measurement may be greater than or less than the first measurement. In some instances, there may be no change between measurements, and this absence of change may affect the therapeutic decision made by a practitioner in some embodiments.

Changes in the concentration of various combinations of biomarkers, such as those of a biomarker panel disclosed herein, will indicate to a practitioner a subject's responder status, i.e., whether or not a subject is a responder or nonresponder to a therapy. It should be appreciated that changes in biomarker concentrations can, in some cases, also indicate various degrees of response to a therapy. Thus, in some embodiments, a subject may be determined to be a strong responder, an intermediate responder or a weak responder. A subject associated with one of these response categories may optionally be given a different therapy compared to a subject associated with another. A practitioner can devise any number of response categories according to his or her needs.

Whether a subject is a responder or nonresponder to a therapy can be determined by the number and/or degree of changes observed in any combination of biomarkers of any biomarker panel disclosed herein. Identifying the responder status, which includes identifying nonresponder status, of a subject can aid the practitioner in choosing an appropriate therapy as discussed below.

One advantage of the biomarker panels of the invention is that they allow a practitioner to detect a response to a therapy, such as administration of a disease-modulating drug, within a short period of time, typically 1, 2, 3, 4, 5, 6 or 7 days, preferably within 1, 2, 3 or 4 days. Responder status can often be determined within 1 day after administration of the drug. Biomarker measurements made within 3 days after administration of the drug can be used to determine if changes in dosage are necessary. It may also be advantageous to detect a response to a therapy within 2, 3 or 4 weeks.

There are numerous ways of determining a subject's tendency to respond to a therapy. In various embodiments, a subject's responder status is based on a change observed for each biomarker of a biomarker panel or of a subset of the biomarker panel. In other words, if a biomarker panel comprises or consists of 9 biomarkers, a subject's responder status may be based on a change observed in 1, 2, 3, 4, 5, 6, 7, 8 or 9 biomarkers, in any combination.

In some embodiments, a change as defined above (e.g. an increase or a decrease, depending on the marker) in at least two markers (for example, selected from ghrelin (e.g. total ghrelin), obestatin, cholecystokinin, GLP-1 (e.g. GLP-1(6-37)-NH₂), NPY and proopiomelanocortin (e.g., α-MSH)) allows calling a patient a “responder”, e.g., someone to whom a drug is beneficial. In alternative embodiments, a change in at least 3, 4, 5, 6, 7, 8 or 9 of the markers allows the continuation of the drug.

The types of changes in the biomarker levels used to indicate a response may vary depending on the type of response being detected. In the case of a subject experiencing hunger followed by satiety after eating or other treatment, plasma or serum levels of ghrelin (e.g. total ghrelin) decrease, obestatin increase, cholecystokinin increase, GLP-1 (e.g. GLP-1(6-37)-NH₂) increase, NPY decrease and proopiomelanocortin (e.g. α-MSH) increase. Thus, in some embodiments, a therapy that comprises inducing a feeling of satiety or suppressing appetite could cause these changes to occur, preferably according to the ranges disclosed herein. Such a therapy may be useful in treating obesity. In one embodiment, all of these changes occurs. In other embodiments, where the goal is to reduce a feeling of satiety or increase appetite, a therapy could be effected that causes changes in the opposite direction to occur. In one embodiment, none of these changes occurs. Other combinations of these changes (i.e. an increase or decrease beyond a reference value) and of changes in other panels could be used to determine a variety of responses.

In some embodiments, measurements of biomarker concentrations may be combined with genotyping of the subject to determine a therapy. That is, by combining biomarker concentrations with a subject's genotype for expressing, for example, a particular member of the CYP superfamily, a practitioner can choose a therapy or dosage accordingly.

Once a practitioner has made a determination, based on the comparison of biomarker concentrations between a first and second measurement, as to whether a subject is a responder, nonresponder or a responder of a certain degree to a therapy (e.g. the administration of a disease-modulating drug), a practitioner may decide to effect a therapy based on this determination.

In some embodiments, the therapy comprises repeating or maintaining a therapy, such as administration of a disease-modulating drug. A practitioner might choose this therapy, if, for example, a subject that is administered a disease-modulating drug according to a first dosage regimen is determined to be a responder based on a change or set of changes described herein. In some embodiments, if the concentrations of all of the biomarkers of a biomarker panel decrease (except for biomarkers that tend to move in the opposite direction compared to others in indicating a response) or otherwise change to indicate a response as described herein compared to a first measurement, then the therapy comprises repeating or maintaining administration of a disease-modulating drug.

In some embodiments, the therapy comprises administering an additional drug to the subject, wherein the additional drug is different from a first administered drug. Other drugs useful in the present invention are described herein. An exemplary additional drug is a statin.

In some embodiments, the therapy comprises discontinuing a therapy, such as administration of a disease-modulating drug. A practitioner might choose this therapy, if, for example, a subject that is administered a disease-modulating drug according to a first dosage regimen is determined to be a nonresponder, e.g., there is no significant change in one or more of the biomarker concentrations. A practitioner might also choose this therapy, if, for example, a subject is a weak responder. For instance, a practitioner might determine that the risks of administering a drug outweighs the benefits of the weak response. In some embodiments, if the concentration of one or more biomarkers does not increase or decrease in a manner indicative of response to a first therapy (such as administration of a disease-modulating drug) as described herein, then a second therapy comprises discontinuing the first therapy.

In some embodiments, a therapy comprises administering a disease modulating drug according to a second dosage regimen. In these embodiments, the second dosage regimen will be different from the first dosage regimen associated with administration of the disease-modulating drug before measurement of a biomarker panel. In exemplary embodiments, the first dosage regimen comprises administering the disease modulating drug at a first dose and the therapy comprises administering the disease modulating drug at a second dose that depends on the degree of change in the expression of MCP-1 nucleic acid, MMP-9 nucleic acid or NFκB nucleic acid (or other nucleic acids of other panels), for example, or in the concentrations of some combination (such as all) of the biomarkers. In some embodiments, the therapy comprises administering a disease-modulating drug according to an adjusted dosage regimen compared to a previous dosage regimen.

The biomarkers of the invention show a statistically significant difference between different responses to a disease-modulating drug. In various embodiments, diagnostic tests that use these biomarkers alone or in combination show a sensitivity and specificity of at least about 85%, at least about 90%, at least about 95%, at least about 98% and about 100%.

The articles “a,” “an” and “the” as used herein do not exclude a plural number of the referent, unless context clearly dictates otherwise. The conjunction “or” is not mutually exclusive, unless context clearly dictates otherwise. The term “include” is used to refer to non-limiting examples.

All references, publications, patent applications, issued patents, accession records and databases cited herein, including in any appendices, are incorporated by reference in their entirety for all purposes.

1. A kit comprising a solid support comprising:
(a) a capture binding ligand selective for total ghrelin,
(b) a capture binding ligand selective for obestatin,
(c) a capture binding ligand selective for cholecystokinin,
(d) a capture binding ligand selective for GLP-1(6-37)-NH₂,
(e) a capture binding ligand selective for NPY and
(f) a capture binding ligand selective for α-MSH.

2. The kit of claim 1 wherein one of the capture binding ligands comprises an antibody.

3. The kit of any preceding claim further comprising:
(a) a soluble binding ligand selective for total ghrelin,
(b) a soluble binding ligand selective for obestatin,
(c) a soluble binding ligand selective for cholecystokinin,
(d) a soluble binding ligand selective for GLP-1(6-37)-NH₂,
(e) a soluble binding ligand selective for NPY and
(f) a soluble binding ligand selective for α-MSH,
wherein each of the soluble capture ligands comprises a detectable label.

4. The kit of claim 3 wherein a detectable label is a fluorophore.

5. The kit of claim 4 wherein a detectable label comprises biotin.

6. The kit of claim 5 further comprising a horseradish peroxidase conjugate.

7. The kit of claim 6 further comprising a precipitating agent.

8.-9. (canceled)

10. A method of treating obesity in a subject comprising:
(a) measuring the concentrations of total ghrelin, obestatin, cholecystokinin, GLP-1(6-37)-NH₂, NPY and α-MSH in a first sample from the subject; and
(b) effecting a first therapy on the subject, wherein one, a combination or all of the concentrations of total ghrelin, obestatin, cholecystokinin, GLP-1(6-37)-NH₂, NPY and α-MSH in a second sample from the subject after a first therapy are changed with respect to the first sample, thereby treating obesity in the subject.

11. The method of claim 10 wherein one, a combination or all of the changes selected from (a) a decrease in total ghrelin concentration, (b) an increase in obestatin concentration, (c) an increase in cholecystokinin concentration, (d) an increase in GLP-1(6-37)-NH₂ concentration, (e) a decrease in NPY concentration and (f) an increase in α-MSH concentration occur(s) between the first sample and the second sample from the subject after the first therapy.

12. The method of claim 11 wherein one, a combination or all of the changes selected from (a) a decrease in total ghrelin concentration to below about 5 μg/L, (b) an increase in obestatin concentration to above about 40 ng/L, (c) an increase in cholecystokinin concentration to above about 1 μg/L, (d) an increase in GLP-1(6-37)-NH₂ concentration to above about 20 pg/mL, (e) a decrease in NPY concentration to below about 10 pmol/L and (f) an increase in α-MSH concentration to above about 20 ng/L occur(s) between the first sample and the second sample from the subject after the first therapy.

13. The method of claim 10 wherein, in the second sample, total ghrelin concentration is below about 5 μg/L, obestatin concentration is above about 40 ng/L, cholecystokinin concentration is above about 1 μg/L, GLP-1(6-37)-NH₂ concentration is above about 20 pg/mL, NPY concentration is below about 10 pmol/L and α-MSH concentration is above about 20 ng/L.

14. The method of claim 10 wherein effecting the first therapy comprises administering a first disease-modulating drug to the subject, optionally wherein the drug is a GLP-1 analog.

15. The method of claim 10 wherein effecting the first therapy comprises causing the subject to follow a dietary regimen having a high fiber and low carbohydrate content.

16. A method of assessing the efficacy of a first therapy on a subject experiencing obesity comprising:
(a) taking a first measurement of the concentrations of total ghrelin, obestatin, cholecystokinin, GLP-1(6-37)-NH₂, NPY and α-MSH in a first sample from the subject;
(b) effecting the first therapy on the subject;
(c) taking a second measurement of the concentrations of total ghrelin, obestatin, cholecystokinin, GLP-1(6-37)-NH₂, NPY and α-MSH in a second sample from the subject after the first therapy; and
(d) making a comparison between the first and second measurements.

17. The method of claim 16 further comprising (e) effecting a second therapy on the subject based on the comparison.

18. The method of claim 17 wherein effecting the first therapy comprises administering a first disease-modulating drug to the subject according to a first dosage regimen.

19. The method of claim 18 wherein effecting the second therapy comprises making a decision regarding the continued administration of the first disease-modulating drug.

20. The method of claim 18 wherein effecting the second therapy comprises administering a second disease-modulating drug to the subject.

21. The method of claim 18 wherein effecting the second therapy comprises administering a statin to the subject.

22. The method of claim 18 wherein effecting the second therapy comprises discontinuing the administration of the first disease-modulating drug.

23. The method of claim 18 wherein effecting the second therapy comprises repeating or maintaining the administration of the first disease-modulating drug.

24. The method of claim 18 wherein effecting the second therapy comprises administering the first disease-modulating drug according to an adjusted dosage regimen compared to the first dosage regimen.

25. The method of claim 24 wherein the adjusted dosage regimen depends on the degree of change in the concentration(s) of one, a combination or all of total ghrelin, obestatin, cholecystokinin, GLP-1(6-37)-NH₂, NPY and α-MSH between the first and second measurement.

26. The method of claim 23 wherein if one, a combination or all of the changes selected from (a) a decrease in total ghrelin concentration, (b) an increase in obestatin concentration, (c) an increase in cholecystokinin concentration, (d) an increase in GLP-1(6-37)-NH₂ concentration, (e) a decrease in NPY concentration and (f) an increase in α-MSH concentration occur(s) between the first and second measurements, then effecting the second therapy comprises repeating or maintaining the administration of the first disease-modulating drug.

27. The method of claim 26 wherein if one, a combination or all of the changes selected from (a) a decrease in total ghrelin concentration to below about 5 (b) an increase in obestatin concentration to above about 40 ng/L, (c) an increase in cholecystokinin concentration to above about 1 μg/L, (d) an increase in GLP-1(6-37)-NH₂ concentration to above about 20 pg/mL, (e) a decrease in NPY concentration to below about 10 pmol/L and (f) an increase in α-MSH concentration to above about 20 ng/L occur(s) between the first and second measurements, then effecting the second therapy comprises repeating or maintaining the administration of the first disease-modulating drug.

28. The method of claim 22 wherein if one, a combination or all of the changes selected from (a) a decrease in total ghrelin concentration, (b) an increase in obestatin concentration, (c) an increase in cholecystokinin concentration, (d) an increase in GLP-1(6-37)-NH₂ concentration, (e) a decrease in NPY concentration and (f) an increase in α-MSH concentration do(es) not occur between the first and second measurements, then effecting the second therapy comprises discontinuing the administration of the first disease-modulating drug.

29. The method of claim 28 wherein if one, a combination or all of the changes selected from (a) a decrease in total ghrelin concentration to below about 5 μg/L, (b) an increase in obestatin concentration to above about 40 ng/L, (c) an increase in cholecystokinin concentration to above about 1 μg/L, (d) an increase in GLP-1(6-37)-NH₂ concentration to above about 20 pg/mL, (e) a decrease in NPY concentration to below about 10 pmol/L and (f) an increase in α-MSH concentration to above about 20 ng/L do(es) not occur between the first and second measurements, then effecting the second therapy comprises discontinuing the administration of the first disease-modulating drug.

30. The method of claim 18 wherein the first disease-modulating drug is a GLP-1 analog.

31. The method of claim 16 wherein effecting the first therapy comprises causing the subject to follow a dietary regimen having a high fiber and low carbohydrate content.

32. The method of claim 31 wherein one, a combination or all of the changes selected from (a) a decrease in total ghrelin concentration, (b) an increase in obestatin concentration, (c) an increase in cholecystokinin concentration, (d) an increase in GLP-1(6-37)-NH₂ concentration, (e) a decrease in NPY concentration and (f) an increase in α-MSH concentration occur(s) between the first and second measurements.

33. The method of claim 32 wherein one, a combination or all of the changes selected from (a) a decrease in total ghrelin concentration to below about 5 μg/L, (b) an increase in obestatin concentration to above about 40 ng/L, (c) an increase in cholecystokinin concentration to above about 1 μg/L, (d) an increase in GLP-1(6-37)-NH₂ concentration to above about 20 pg/mL, (e) a decrease in NPY concentration to below about 10 pmol/L and (f) an increase in α-MSH concentration to above about 20 ng/L occur(s) between the first and second measurements.

34. The method of claim 16 wherein one, a combination or all of the changes selected from (a) a decrease in total ghrelin concentration, (b) an increase in obestatin concentration, (c) an increase in cholecystokinin concentration, (d) an increase in GLP-1(6-37)-NH₂ concentration, (e) a decrease in NPY concentration and (f) an increase in α-MSH concentration do(es) not occur between the first and second measurements.

35. The method of claim 34 wherein one, a combination or all of the changes selected from (a) a decrease in total ghrelin concentration to below about 5 μg/L, (b) an increase in obestatin concentration to above about 40 ng/L, (c) an increase in cholecystokinin concentration to above about 1 μg/L, (d) an increase in GLP-1(6-37)-NH₂ concentration to above about 20 pg/mL, (e) a decrease in NPY concentration to below about 10 pmol/L and (f) an increase in α-MSH concentration to above about 20 ng/L do(es) not occur between the first and second measurements.

36. The method of claim 31 further comprising effecting a second therapy comprising administering a disease modulating drug to the subject, optionally wherein the drug is a GLP-1 analog.

37.-38. (canceled)

39. The method of claim 16 wherein a sample is contacted with a solid support comprising:
(a) a capture binding ligand selective for total ghrelin,
(b) a capture binding ligand selective for obestatin,
(c) a capture binding ligand selective for cholecystokinin,
(d) a capture binding ligand selective for GLP-1(6-37)-NH₂.
(e) a capture binding ligand selective for NPY, and
(f) a capture binding ligand selective for α-MSH.

40. A method of acquiring data relating to a sample comprising (a) taking a measurement of the concentrations of total ghrelin, obestatin, cholecystokinin, GLP-1(6-37)-NH₂, NPY and α-MSH in the sample, thereby acquiring data relating to the sample.

41. The method of claim 40 wherein the sample is derived from a subject, optionally wherein the subject is experiencing obesity.

42. (canceled)

43. The method of claim 40 wherein the sample is contacted with a solid support comprising:
(a) a capture binding ligand selective for total ghrelin,
(b) a capture binding ligand selective for obestatin,
(c) a capture binding ligand selective for cholecystokinin,
(d) a capture binding ligand selective for GLP-1(6-37)-NH₂,
(e) a capture binding ligand selective for NPY, and
(f) a capture binding ligand selective for α-MSH.

44. (canceled)

45. Use of the kit of claim 1 to determine whether a subject belongs to a population that would benefit from a second therapy, wherein the subject has undergone a first therapy.

46. The use of claim 45 comprising
(a) contacting a first sample from the subject with the solid support of the kit;
(b) taking a first measurement of the concentrations of total ghrelin, obestatin, cholecystokinin, GLP-1(6-37)-NH₂, NPY and α-MSH in the first sample;
(c) effecting a first therapy on the subject;
(d) contacting a second sample from the subject with the solid support of the kit;
(e) taking a second measurement of the concentrations of total ghrelin, obestatin, cholecystokinin, GLP-1(6-37)-NH₂, NPY and α-MSH in the second sample; and
(f) making a comparison of the first and second measurements.

47. The use of claim 46 wherein the first therapy comprises administering a first disease-modulating drug to the subject according to a first dosage regimen.

48. The use of claim 47 wherein the second therapy comprises administering a second disease-modulating drug to the subject.

49.-66. (canceled)
